SEQUENCE LISTING (9 sequences)

SEQ ID NO: 1 - protein, 133 aa, Homo sapiens [sequence omitted]

SEQ ID NO: 2 - protein, 213 aa, Homo sapiens
EIVLTQSPATLSLSPGERVTLTCSASSSVSYMLWYQQKPGSSPKPWIFDTSNLASGFPARFSGSGSGTSYSLIISSMEAEDAATYYCHQRSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Homo sapiens
QIQLVQSGAEVKKPGETVKISCKASGYTFTNYGMNWVKQTPGKGLKWMGWINTYTGEPTYADDFKGRFAFSLETSTSTAFLQINNLRSEDTATYFCVRFISKGDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 579 aa, Artificial Sequence
  KS-ala-IL2 Heavy Chain Amino Acid Sequence
QIQLVQSGAEVKKPGETVKISCKASGYTFTNYGMNWVKQTPGKGLKWMGWINTYTGEPTYADDFKGRFAFSLETSTSTAFLQINNLRSEDTATYFCVRFISKGDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - protein, 229 aa, Artificial Sequence
  dI-NHS76 Light Chain
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKVTVLGGHQDSDPLPLIHPAGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 6 - protein, 580 aa, Artificial Sequence
  dI-NHS76(gamma2h)(FN>AQ)-ala-IL2 Heavy Chain Amino Acid Sequence
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSATATPGAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 220 aa, Homo sapiens
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 574 aa, Artificial Sequence
  Hu14.18 IgG1-IL2 Heavy Chain Amino Acid Sequence
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTL

SEQ ID NO: 9 - protein, 1032 aa, Artificial Sequence [sequence omitted]

(19) United States
(12) Patent Application Publication
(10) Pub. No.: US 2013/0017168 A1
(43) Pub. Date: Jan. 17, 2013

(54) METHODS FOR ENHANCING THE EFFICACY OF IL-2 MEDIATED IMMUNE RESPONSES

(71) Applicants: Stephen D. Gillies, Carlisle, MA (US); Kin-Ming Lo, Lexington, MA (US); Yan Lan, Belmont, MA (US)

(72) Inventors: Stephen D. Gillies, Carlisle, MA (US); Kin-Ming Lo, Lexington, MA (US); Yan Lan, Belmont, MA (US)

(73) Assignee: Merck Patent GmbH, Darmstadt (DE)

(21) Appl. No.: 13/434,354

(22) Filed: Mar. 29, 2012

Related U.S. Application Data
(63) Continuation of application No. 11/825,220, filed on Jul. 5, 2007, now abandoned.
(60) Provisional application No. 60/818,741, filed on Jul. 6, 2006.
(60) Provisional application No. 60/856,139, filed on Nov. 2, 2006.

(51) Int. Cl. A61K 38/20; A61P 35/00; A61P 37/04

(52) U.S. Cl. 424/85.2

(57) ABSTRACT

Methods directed to enhancing the effectiveness of IL-2 in stimulating the immune system is disclosed. According to one method, an antagonist directed against the CD25 subunit of the high-affinity IL-2 receptor complex is administered in conjunction with IL-2. The CD25 antagonist may be an anti-CD25 antibody. According to another method, an anti-IL-2 antibody is administered in conjunction with IL-2. In another method, a mutant IL-2 with diminished ability to bind the CD25 subunit of the high-affinity IL-2 receptor complex is administered. In another method, an CD4 antagonist is administered in conjunction with IL-2 in order to stimulate the immune system.

### CROSS-REFERENCE TO RELATED APPLICATIONS

This application claims priority to and the benefit of U.S. Provisional Patent Application No. 60/818,741, filed Jul. 6, 2006, and U.S. Provisional Patent Application No. 60/856,139, filed Nov. 2, 2006, the disclosures of each of which are incorporated by reference herein.

### FIELD OF THE INVENTION

The invention relates generally to methods for enhancing IL-2 mediated immune responses. More specifically, the invention relates to methods using CD25 antagonists, such as, for example, an anti-CD25 antibody, or a CD4 antagonist, such as an anti-CD4 antibody, to enhance the efficacy of IL-2 therapy.

### BACKGROUND OF THE INVENTION

It is useful to stimulate the immune system of mammals suffering from a viral infection or tumor growth towards an adaptive cell mediated immune response, which has evolved to clear intracellular pathogens. An important population of immune cells that are thereby activated are the CD8+ effector T-cells (cytotoxic lymphocytes). It is well known in the art that IL-2 stimulates a wide variety of immune cells, including monocytes, NK cells and T-cells. IL-2 is used in the clinic to stimulate a cell mediated immune response, and is approved by the FDA for standard therapy in patients with metastatic melanoma or metastatic kidney cancer (e.g., aldesleukin (Chiron), also known as Proleukin®).

The repertoire of T-cells involved in a cell mediated adaptive immune response include CD8+ memory T-cells, CD8+ effector T-cells and regulatory T-cells (T_{regs}). These T_{regs} play an important role in the adaptive immune program by dampening the activity of effector and memory T-cells. It has been observed, however, that IL-2 also activates the T_{reg} subset of T-cells, which then can act to suppress CD8+ T-cells, or to tolerize other T-cells. Thus, IL-2 is involved in both the activation of the adaptive immune response and its attenuation.

L_{reg} cells are characterized by the expression of CD4 and the transcription factor FoxP3, which in turn activates the expression of CD25, the α subunit of the IL-2 receptor complex (CD4+CD25+ cells). Thus, CD25 is constitutively expressed in T_{reg} cells. Association of CD25 with the signaling components of the IL-2 receptor complex (the β subunit CD122 and the γ subunit CD 132) converts the intermediate-affinity IL-2 receptor complex into a high-affinity IL-2 receptor complex. IL-2 activation of T_{reg} cells occurs through a signaling pathway relayed by the high-affinity IL-2 receptor complex.

High level CD25 expression is a characteristic of activated T-cells, making these cells responsive to IL-2 via the high-affinity IL-2 receptor complex. Therapies based on the blockade of CD25 have been developed with the rationale that they will inhibit IL-2 mediated signaling in activated T-cells and have immunosuppressive effects. Anti-CD25 antibodies, such as daclizumab (Roche), also known as Zenapax®, and basiliximab (Novartis), also known as Simulect®, have been approved by the FDA for the prevention of acute organ rejection following kidney transplantation.

Because of the dual role of IL-2, there remains a need in the art to provide more efficacious IL-2-mediated therapies.

### SUMMARY OF THE INVENTION

According to one aspect, the invention is a method of enhancing the immunostimulatory effect of IL-2 in a patient. The method includes the steps of administering a CD25 antagonist and a protein having an IL-2 moiety. The CD25 antagonist is administered in an amount effective to enhance the immunostimulatory effect of the protein comprising an IL-2 moiety. The IL-2 is, for example, in one embodiment, mature human IL-2. In one embodiment, the patient is, for example, a human. In a further embodiment, the protein having the IL-2 moiety is capable of activating an intermediate-affinity IL-2 receptor complex.

According to the invention, in one embodiment, the method of enhancing the immunostimulatory effect of IL-2 in a patient is for treating cancer, while in another embodiment, the method treats a viral infection.

In another embodiment, the protein having an IL-2 moiety has a second IL-2 moiety. In a further embodiment, the protein having a second IL-2 moiety further includes an immunoglobulin moiety. In one embodiment, the immunoglobulin moiety is an Fc moiety. In yet another embodiment, the immunoglobulin moiety is an antibody. In an even further embodiment, the antibody has a variable region directed to an antigen presented on a tumor cell.

In yet another embodiment, the antibody has a variable region directed to an antigen present in a tumor cell environment. In an alternate embodiment, the antigen present in the tumor cell environment is present in a higher concentration than in a normal cell environment.

In another embodiment according to the invention, the CD25 antagonist is an anti-CD25 antibody, or a portion thereof capable of binding to CD25. The anti-CD25 antibody is daclizumab in one embodiment, while in another embodiment, the anti-CD25 antibody is basiliximab.

In a further embodiment, the CD25 antagonist is a protein that binds to the surface of IL-2 and inhibits the interaction between IL-2 and the CD25 subunit of the IL-2 high-affinity receptor. In a further embodiment, CD25 antagonist is an antibody, for example, an anti-IL-2 antibody or portion thereof.

According to an embodiment of the invention, the CD25 antagonist is administered prior to administration of the protein having an IL-2 moiety, while in another embodiment, the CD25 antagonist is administered contemporaneously with the protein having an IL-2 moiety. In a further embodiment, an anti-cancer vaccine is administered in conjunction with the anti-CD25 antibody and the protein having an IL-2 moiety. For example, the anti-cancer vaccine is administered prior to the anti-CD25 antibody and the protein having an IL-2 moiety in one embodiment, while in another embodiment, the anti-cancer vaccine is administered after the administration of the anti-CD25 antibody but before the administration of the protein having an IL-2 moiety. Alternately, the anti-cancer vaccine is administered after the administration of both the anti-CD25 antibody and the protein having an IL-2 moiety. According to another embodiment, the method further includes administration of an immunostimulator in addition to the protein comprising an IL-2 moiety.

In another embodiment, the protein comprising an IL-2 moiety is capable of activating an intermediate-affinity IL-2 receptor complex, while in another embodiment, the IL-2 moiety is not capable of activating a high-affinity IL-2 receptor complex. In yet another embodiment, the protein comprising an IL-2 moiety is capable of binding the β-subunit of an IL-2 receptor complex, but is not capable of binding the α-receptor subunit of an IL-2 receptor complex.

According to the invention, in one embodiment, an effective amount of the CD25 antagonist is between about 0.1 mg/kg and 10 mg/kg per dose, while in another embodiment, the effective amount of CD25 antagonist is between about 0.5 mg/kg and 2 mg/kg per dose. In yet a further embodiment, the effective amount of CD25 antagonist is about 1 mg/kg per dose.

In another embodiment, the effective amount of the protein comprising an IL-2 moiety is between, for example, about 0.004 mg/m² and 4 mg/m², while in another embodiment, the effective amount of the protein comprising an IL-2 moiety is between about 0.12 mg/m² and 1.2 mg/m².

According to another embodiment, the invention includes a method of stimulating effector cell function in a patient. The method comprises the step of administering to a patient an IL-2 fusion protein and an inhibitor of the interaction between IL-2 and IL-2 receptor α subunit. The inhibitor is administered in an amount effective to enhance the immunostimulatory effect of the IL-2 fusion protein. In one embodiment, the inhibitor is an anti-IL-2 antibody. In another embodiment, the anti-IL-2 antibody is directed against at least the portion of IL-2 necessary for binding to the α subunit of the IL-2 high-affinity receptor. In a further embodiment, the inhibitor does not affect the ability of IL-2 from binding with the β subunit of an IL-2 receptor.

In a further embodiment, the invention includes another method of stimulating effector cell function in a patient. For example, in one embodiment, the method includes administering to a patient an IL-2 fusion protein containing one or more mutations that reduce or abolish the interaction between IL-2 and the IL-2 receptor α subunit. The IL-2 fusion protein is administered in an amount effective to stimulate effector cell function. In a further embodiment, the IL-2 fusion protein contains mutations in the IL-2 moiety corresponding to residues R38 and F42 of wild-type human IL-2. According to another embodiment, the one or more mutations reduce or abolish the interaction between the portion of the IL-2 moiety of the IL-2 fusion protein necessary for binding to the α subunit of the IL-2 high-affinity receptor and the α subunit of the IL-2 high-affinity receptor.

According to another aspect, the invention includes a pharmaceutical composition including an IL-2 fusion protein and a protein that binds to IL-2. The protein that binds to IL-2 blocks the interaction between IL-2 and the IL-2 receptor α subunit. In one embodiment, the protein that binds to IL-2 is an anti-IL2 antibody. In another embodiment, the protein that binds to IL-2 does not block the interaction between IL-2 and a β subunit of an IL-2 high or intermediate-affinity receptor. For example, the protein that binds to IL-2 and does not block the interaction between IL-2 and a β subunit of an IL-2 high or intermediate-affinity receptor is an anti-IL-2 antibody directed against only the portion of IL-2 necessary for binding to the α subunit of the high-affinity IL-2 receptor.

According to another embodiment, the invention includes a pharmaceutical composition comprising an IL-2 fusion protein containing one or more mutations that reduce or abolish the interaction between IL-2 and the IL-2 receptor α subunit. In another embodiment, the invention includes a pharmaceutical composition comprising an anti-CD25 antibody and a protein comprising an IL-2 moiety, while in another embodiment, the pharmaceutical composition comprises an IL-2 fusion protein and a protein that binds to IL-2. In yet another embodiment, the pharmaceutical composition comprises an IL-2 fusion protein and an inhibitor of the interaction between IL-2 and an IL-2 receptor α subunit.

In a further embodiment, methods according to the invention are useful for enhancing the efficacy of a vaccine administered to a patient. According to the invention, the vaccine can be an anti-cancer vaccine, or a vaccine directed against any other condition for which a vaccine is suitable. In one embodiment, the method of enhancing the efficacy of a vaccine includes administering to a patient an antigen of the vaccine as well as an IL-2 fusion protein containing one or more mutations that reduce or abolish the interaction between IL-2 and the IL-2 receptor α subunit. In another embodiment, the method includes the steps of administering to a patient an antigen of the vaccine as well as a nucleic acid encoding an IL-2 fusion protein containing one or more mutations that reduce or abolish the interaction between IL-2 and the IL-2 receptor α subunit.

In another embodiment, a method of enhancing the efficacy of a vaccine includes administering to a patient an antigen of the vaccine, an IL-2 fusion protein, and a protein that binds IL-2. In another embodiment, the method includes administering to a patient a vaccine, a protein that binds IL-2, and a nucleic acid encoding an IL-2 fusion protein. According to yet another embodiment, a method of enhancing the efficacy of a vaccine includes administering to a patient an antigen of the vaccine, an IL-2 fusion protein, and an inhibitor of the interaction between IL-2 and an IL-2 receptor α subunit. According to a further embodiment, a method of enhancing the efficacy of a vaccine includes administering to a patient an antigen of the vaccine, a nucleic acid encoding an IL-2 fusion protein, and an inhibitor of the interaction between IL-2 and an IL-2 receptor α subunit.

In another aspect, the invention includes a method of enhancing the immunostimulatory effect of IL-2 in a patient. The method includes the steps of administering a CD4 antagonist and a protein comprising an IL-2 moiety. The CD4 antagonist is administered in amount effective to enhance the immunostimulatory effect of the protein comprising an IL-2 moiety. The method may alternately include the step of administering an anti-CD25 antagonist. Accordingly, in one embodiment, the anti-CD25 antagonist and the anti-CD4 antagonist are administered prior to the administration of the protein comprising an IL-2 moiety. In another embodiment, the anti-CD25 antagonist and the anti-CD4 antagonist are administered simultaneously. According to the invention, in one embodiment, the anti-CD4 antagonist is an anti-CD4 antibody and the anti-CD25 antagonist is an anti-CD25 antibody.

The invention also includes a protein composition comprising an anti-CD4 antagonist and a protein comprising IL-2. For example, in one embodiment, the composition is of an anti-CD4 antibody and an antibody-IL2 fusion protein. In a further embodiment, the protein composition also comprises an anti-CD25 antagonist, for example, an anti-CD25 antibody.

### BRIEF DESCRIPTION OF THE DRAWINGS

FIG. 1A represents a schematic of the experimental protocol in Example 1, discussed below.

FIG. 1B represents a bar graph of the amounts of CD4+ cells (black bars) and CD8+ (white bars) in a mouse blood sample taken at day 8 from mice treated either with PBS, the anti-CD25 antibody PC61, the combination of PC61 and KS-ala-IL2, and the combination of PC61 and rhIL-2 (recombinant wild-type human IL-2).

FIG. 1C represents a bar graph of percent of total spleen cells comprised by CD4+ cells (black bars) and CD8+ (white bars) in a mouse sample taken at day 8 from mice treated either with PBS, the anti-CD25 antibody PC61, the combination of PC61 and KS-ala-IL2, and the combination of PC61 and rhIL-2.

FIG. 1D represents a bar graph of percent of total spleen cells comprised by CD25+ cells (black bars) and CD4+CD25+ cells (white bars) in a mouse sample taken at day 8 from mice treated either with PBS, the anti-CD25 antibody PC61, the combination of PC61 and KS-ala-IL2, and the combination of PC61 and rhIL-2.

FIG. 2A represents a bar graph of the number of CD8+ cells in a mouse blood sample taken on day 8 (black bars), day 10 (white bars), day 14 (grey bars), and day 21 (striped bars) of mice treated either with PBS, the anti-CD25 antibody PC61, a single dose of KS-ala-IL2 (IC(1)), two doses of KS-ala-IL2 (IC(2)), and the combination of PC61 with a single dose or two doses of KS-ala-IL2.

FIG. 2B represents a bar graph of the number of CD4+CD25+ cells in a mouse blood sample taken on day 8 (black bars), day 14 (white bars), and day 21 (grey bars) of mice treated either with PBS, the anti-CD25 antibody PC61, a single dose of KS-ala-IL2 (IC(1)), two doses of KS-ala-IL2 (IC(2)), and the combination of PC61 with a single dose or two doses of KS-ala-IL2.

FIG. 2C represents a bar graph of the fractional number of immune cells in the blood relative to PBS-treated controls for CD4+ cells (black bars), CD8+ (white bars), and NK1.1+ cells (grey bars) at day 8 of mice treated either with PBS, the anti-CD25 antibody PC61, a single dose of KS-ala-IL2 (IC(1)), two doses of KS-ala-IL2 (IC(2)), and the combination of PC61 with a single dose or two doses of KS-ala-IL2.

FIG. 2D represents a bar graph of the number of CD8+ cells (black bars), memory CD8+ (white bars), and naïve CD8+ cells (hatched bars) in a mouse blood sample taken at day 10 of mice treated either with PBS, the anti-CD25 antibody PC61, a single dose of KS-ala-IL2 (IC(1)), two doses of KS-ala-IL2 (IC(2)), and the combination of PC61 with a single dose or two doses of KS-ala-IL2.

FIG. 2E is a flow cytometry diagram from which the data in FIGS. 2A-D were drawn.

FIGS. 3A-C represent bar graphs of the cell count for CD4 (FIG. 3A), CD8 (FIG. 3B) and NK1.1 (FIG. 3C) cells in peripheral blood samples taken from mice treated in Example 3 below, while FIGS. 3D-F represent bar graphs of percentage of CD4 (FIG. 3D), CD8 (FIG. 3E) and NK1.1 (FIG. 3F) cells in the spleens of the same populations of mice.

FIG. 4A represents a bar graph of the percentage of total spleen cells taken from mice treated according to Example 3, discussed below, that are also CD25+FoxP3+. FIG. 4B is a flow cytometry diagram from which the data in FIG. 4A is drawn.

FIGS. 5A-C refer to Example 4, discussed below. FIG. 5A represents a bar graph of the number of CD4+ cells in a mouse blood sample taken on day 8 from mice subjected to the following treatment: (a) rat IgG antibody in combination with PBS, (b) rat IgG antibody in combination with KS-ala-IL2, (c) rat IgG antibody in combination with KS-ala-monoIL2, (d) rat IgG antibody in combination with KS-ala-IL2(D20T), and (e) rat IgG antibody in combination with KS-murineIL2, (a′) anti-CD25 antibody PC61 in combination with PBS, (b′) anti-CD25 antibody PC61 in combination with KS-ala-IL2, (c′) anti-CD25 antibody PC61 in combination with KS-ala-monoIL2, (d′) anti-CD25 antibody PC61 in combination with KS-ala-IL2(D20T), and (e′) anti-CD25 antibody PC61 in combination with KS-murineIL2. The data represents the mean from n=3 mice, with standard deviation.

FIG. 5B represents a bar graph of the number of CD8+ cells in a mouse blood sample taken on day 8 from mice subjected to the following treatment: (a) rat IgG antibody in combination with PBS, (b) rat IgG antibody in combination with KS-ala-IL2, (c) rat IgG antibody in combination with KS-ala-monoIL2, (d) rat IgG antibody in combination with KS-ala-IL2(D20T), and (e) rat IgG antibody in combination with KS-murineIL2, (a′) anti-CD25 antibody PC61 in combination with PBS, (b′) anti-CD25 antibody PC61 in combination with KS-ala-IL2, (c′) anti-CD25 antibody PC61 in combination with KS-ala-monoIL2, (d′) anti-CD25 antibody PC61 in combination with KS-ala-IL2(D20T), and (e′) anti-CD25 antibody PC61 in combination with KS-murineIL2. The data represents the mean from n=3 mice, with standard deviation.

FIG. 5C represents a bar graph of the number of NK1.1+ cells in a mouse blood sample taken on day 8 from mice subjected to the following treatment: (a) rat IgG antibody in combination with PBS, (b) rat IgG antibody in combination with KS-ala-IL2, (c) rat IgG antibody in combination with KS-ala-monoIL2, (d) rat IgG antibody in combination with KS-ala-IL2(D20T), and (e) rat IgG antibody in combination with KS-murineIL2, (a′) anti-CD25 antibody PC61 in combination with PBS, (b′) anti-CD25 antibody PC61 in combination with KS-ala-IL2, (c′) anti-CD25 antibody PC61 in combination with KS-ala-monoIL2, (d′) anti-CD25 antibody PC61 in combination with KS-ala-IL2(D20T), and (e′) anti-CD25 antibody PC61 in combination with KS-murineIL2. The data represents the mean from n=3 mice, with standard deviation.

FIGS. 6A-E represent bar graphs of cell counts for CD4 (FIG. 6A), CD4+CD25+ (FIG. 6B), CD8 (FIG. 6C), CD8+CD25+ (FIG. 6D), and NK1.1 (FIG. 6E) cells present in peripheral blood taken from mice treated according to the protocol described in Example 9 below.

FIG. 7 is a depiction of data of percent surface metastases and tumor burden for mice transfected with B16 melanoma cells and treated according to the protocol described in Example 7 below.

FIGS. 8A-B represent bar graphs of cell counts in peripheral blood samples taken from SCID mice treated as described in Example 10 below. FIG. 8A represents counts for DX5+ NK cells (black bars) and DX5+CD11b+NK cells (white bars). FIG. 8B represents counts of Gr1+ granulocytes. FIGS. 8C-D represent bar graphs of cell counts in peripheral blood samples taken from B1/6 mice. FIG. 8C represents cell counts for CD8+ cells, while FIG. 8D represents NK1.1+ cell counts.

FIG. 9 is a depiction of data relating to the phenotype of CD4 cells present in the peripheral blood and spleen of mice treated according to the protocol described in Example 13. In particular, the data address the percentage of CD4 cells that were CD25+FOXP3+.

FIGS. 10A-F represent bar graphs of cell counts in blood samples taken from mice treated according to the protocol described in Example 13. CD4 cell counts are depicted in FIG. 10A; CD4+CD25+ cell counts are depicted in FIG. 10B; CD8 cell counts are depicted in FIG. 10C; CD8+CD25+ cell counts are depicted in FIG. 10D; NK1.1 cell counts are depicted in FIG. 10E; and Gr1 cell counts are depicted in FIG. 10F.

FIG. 11 represents the mature human IL-2 amino acid sequence (SEQ ID NO:1).

FIG. 12 represents the light chain amino acid sequence for the KS antibody (SEQ ID NO:2).

FIG. 13 represents the heavy chain amino acid sequence for the KS antibody (SEQ ID NO:3).

FIG. 14 represents the heavy chain amino acid sequence for the KS-ala-IL2 antibody fusion protein (SEQ ID NO:4). KS-ala-IL2 means that the heavy chain of the KS antibody is fused to IL-2 and the C-terminal lysine of the antibody portion is substituted with an alanine residue.

FIG. 15 represents the light chain amino acid sequence for the deimmunized NHS76 antibody (SEQ ID NO:5).

FIG. 16 represents the heavy chain amino acid sequence for the deimmunized NHS76 antibody fused to IL2 called NHS76(γ2h)(FN>AQ)-ala-IL2 (SEQ ID NO:6), wherein the heavy chain has an IgG2 hinge with other domains from IgG1, the C-terminal lysine of heavy chain is substituted with alanine, and the sequence of phenylalanine asparagine is changed to alanine glutamine.

FIG. 17 represents the light chain amino acid sequence for the human 14.18 IgG1 antibody (SEQ ID NO:7).

FIG. 18 represents the heavy chain amino acid sequence for the human 14.18 IgG1 antibody fused to IL2, with the C-terminal lysine of the antibody deleted (SEQ ID NO:8).

FIG. 19 represents the mature human CEA-Fc-IL2 (SEQ ID NO:9) amino acid sequence which is the antigen CEA fused to the N-terminus of an Fc portion. The C-terminus of the Fc-portion is fused to IL-2.

### DETAILED DESCRIPTION OF THE INVENTION

One of the major challenges of treating cancer with immune therapies is the need to promote anti-tumor activity without simultaneously activating the regulatory systems of the immune system designed to control immune system activation. According to the invention, ways of releasing cytotoxic CD8+ T cell proliferation in response to IL-2 from the control of CD25+ T_{regs} inhibition are disclosed. Such mechanisms for reducing or eliminating T_{reg} inhibition include blocking the CD25 receptor on the cell surface of T_{regs} and/or depleting CD4+ cells. Also, another mechanism for achieving the same result is mutation of IL-2 to reduce or eliminate binding with CD25 receptors.

Blocking the CD25 receptor on the cell surface of T_{regs} and/or CD4+ cells, coupled with administration of an IL-2 immunocytokine, or alternatively administering an IL-2 immunocytokine with a mutant IL-2 moiety that has reduced or eliminated binding to CD25 results in a dramatic increase in CD8+ T cell proliferation that far exceeds the level observed when wild-type IL-2 is administered. When the approach includes blockade or a lack of triggering of cell surface CD25, e.g., by mutating IL-2, proliferation occurs in additional immune cell types bearing the intermediate IL-2 receptor, most notably NK cells and granulocytes.

In mammals suffering from a viral infection or tumor growth, it is useful to increase the number of activated T-cells, such as CD8+ cytolytic T-cells (CTLs), and/or NK cells. T-cells and NK cells are generally responsive to IL-2 stimulation. The invention provides for methods that enhance the efficacy of IL-2 treatment in a mammal.

In one aspect of the invention, a method is provided that is more effective than IL-2 alone in stimulating CD8+ and/or NK cells in a mammal. According to one embodiment, the method leads to the expansion of CD8+ cells and NK cells, while T_{reg} cells remain functionally inactivated. According to one embodiment, the method includes administering an CD25 receptor antagonist and a protein composition containing IL-2 (referred to herein as IL-2 protein composition). In one embodiment, the antagonist of the CD25 receptor and the IL-2 protein composition are administered at the same time to a patient, while in another embodiment, the antagonist of the CD25 receptor is administered at a different time than the IL-2 protein composition.

According to another embodiment of the invention, the method includes administering a CD25 receptor antagonist and an IL-2 protein composition containing a mutated version of IL-2. For example, in one embodiment, the IL-2 includes one or more mutations to reduce or eliminate IL-2 binding to the IL-2 α subunit (CD25+) of the high-affinity IL-2 receptor. For example, in one embodiment, the IL-2 moiety includes one or more mutations that reduce or eliminate the ability of at least a portion of the IL-2 moiety to bind to the α subunit (CD25+) of the high-affinity IL-2 receptor. In another embodiment, the IL-2 moiety is an IL-2 fusion protein. For example, in one embodiment, the fusion protein is an antibody fused to an IL-2 moiety.

In a further embodiment, the method includes administering a protein composition containing a mutated version of IL-2. For example, in one embodiment, the mutated version of IL-2 includes one or more mutations to reduce or eliminate the ability of IL-2 to bind to the IL-2 α subunit (CD25+) of the high-affinity IL-2 receptor. The mutated version of IL-2, according to one embodiment, is an IL-2 fusion protein. In a further embodiment, the method includes administering a protein composition containing a mutated version of IL-2 without administering a CD25 receptor antagonist at any point during treatment of the patient with the IL-2 protein composition.

In one embodiment, one or more of the following residues corresponding to positions in wild-type IL-2 are mutated to reduce or eliminate binding between the portion of IL-2 necessary for binding to the IL-2 α subunit and the IL-2 α subunit (CD25+) of the high-affinity IL-2 receptor: R38, F42, K35, M39, K43, or Y45. According to the invention, a mutation may include a deletion, an insertion, or a substitution. In one embodiment, the residue at R38 is replaced with the amino acid residue A, E, N, F, S, L, G, Y or W. In a further embodiment, the residue at M39 is replaced with the amino acid L. In another embodiment, the residue at F42 is replaced with the amino acid residue A, K, L, S, Q, while in yet another embodiment, the residue at K35 is replaced with the amino acid E or A. In an even further embodiment, the amino acid residue at position K43 is replaced with the amino acid E. These mutations are exemplary and any mutation that would adversely affect the binding between IL-2 and the α subunit of the IL-2 high-affinity receptor is contemplated by the invention. According to a further embodiment of the invention, a mutation to IL-2 to reduce or eliminate binding between the portion of IL-2 necessary for binding to the IL-2 α subunit and the IL-2 α subunit (CD25+) of the high-affinity IL-2 receptor does not eliminate binding between IL-2 and the (3 subunit of the high or intermediate-affinity IL-2 receptor.

A reduction or elimination of binding, in one embodiment, refers to a reduction or elimination of binding affinity of one protein for a target as compared to the binding affinity of a reference protein for the target. In one embodiment, the reference protein is a wild-type protein while the protein with reduced or eliminated binding affinity is a mutant. For example, in one embodiment, a mutation to the IL-2 moiety of an IL-2 immunoglobulin fusion protein reduces or eliminates binding affinity of that protein for the IL-2 α subunit as compared to the binding affinity of reference protein. The reference protein is an IL-2 immunoglobulin fusion protein having a wild-type IL-2 moiety.

According to one embodiment, the mutant IL-2 contains only one mutation that affects IL-2Rα subunit binding. For example, in one embodiment the mutant IL-2 contains the mutation R38W. In another embodiment, the mutant IL-2 contains the mutation F42K. In a further embodiment, the mutant IL-2 contains two or more mutations that affect IL-2 binding to the IL-2Rα subunit. For example, the mutant IL-2 contains at least the mutations R38W and F42K.

In another embodiment, a method according to the invention is a method of stimulating effector cell function. For example, according to one embodiment, an IL-2 protein composition and an inhibitor of the interaction between IL-2 and the α subunit of the IL-2 high-affinity receptor are administered to a patient. In one embodiment, the IL-2 protein composition includes a fusion protein. In another embodiment, the inhibitor of the interaction between IL-2 and IL-2 receptor α is an anti-IL-2 antibody directed against the portion of IL-2 necessary for binding to the α subunit of the IL-2 high-affinity receptor, for example, an anti-IL2Rα antibody.

In another embodiment, a method according to the invention for stimulating effector cell function in a patient includes administering to a patient an IL-2 protein composition containing an IL-2 fusion protein. In one embodiment, the IL-2 fusion protein contains one or more mutations in the IL-2 moiety of the fusion protein that reduce or abolish the interaction between the IL-2 moiety and the α subunit of the IL-2 high-affinity receptor. In a further embodiment, the mutation to the IL-2 moiety does not interfere with the interaction between the IL-2 moiety and the β subunit of the IL-2 high-affinity or intermediate-affinity receptor such that binding to the β subunit is maintained. In a further embodiment, an IL-2 fusion protein having a mutation in the IL-2 moiety of the fusion protein that reduces or abolishes binding between the IL-2 moiety and the α subunit of the IL-2 high-affinity receptor is administered to a patient and no CD25 receptor antagonist is administered. In another embodiment, the IL-2 fusion protein is an antibody-IL-2 fusion protein.

According to one embodiment of the invention, the CD25 receptor antagonist is an anti-CD25 antibody. According to a further embodiment, the CD25 receptor antagonist is an antibody specific for the human CD25 protein, for example, in treating a human patient. Examples of anti-CD25 antibodies for use in humans according to the invention include daclizumab and basiliximab. However, other anti-CD25 antibodies are also useful according to the invention. For example, in one embodiment, anti-CD25 antibodies that lack ADCC or CDC effector functions are used, while in another embodiment, derivatives of antibodies such as anti-CD25 small chain variable fragments (scFvs), minibodies, or diabodies directed against CD25 are used according to the invention. Such molecules can be made according to techniques known in the art (see, e.g., Holliger et al., (2005), Nature Biotech., 23(9):1126-1136). Other anti-CD25 antibodies can be created according to methods known to one of skill in the art.

In another embodiment, a method according to the invention for stimulating T cell proliferation includes administering a CD4 antagonist and an IL-2 protein composition. In one embodiment, the CD4 antagonist is administered prior to the administration of the IL-2, protein composition, while in another embodiment, the CD4 antagonist is administered concurrently with the IL-2 protein composition. In yet another embodiment, a method according to the invention for stimulating T cell proliferation includes administering a CD4 antagonist, a CD25 antagonist, and an IL-2 protein composition. For example, in one embodiment a patient is first administered a combination of a CD4 antagonist and CD25 antagonist, followed by administration of an IL-2 protein composition.

It is further contemplated by the invention that while many embodiments of the invention as described herein involve administration of a CD25 antagonist, a CD4 antagonist can be administered in place of the CD25 antagonist according to the invention. Alternatively, a CD25 antagonist can be coadministered with the CD4 antagonist in one embodiment. CD4 antagonists can be administered according to the same dosage schedules as outlined herein for administration of CD25 antagonists.

According to one embodiment of the invention, a CD4 antagonist is an anti-CD4 antibody. In a preferred embodiment, the CD4 antagonist is an anti-CD4 antagonist is an anti-CD4 antibody capable of depleting CD4+ cells. In a particular embodiment, a CD4 antagonist is specific for human CD4. For example, zanolimumab is one example of a human anti-CD4 antibody specific for human CD4. According to one embodiment of the invention, a human anti-CD4 antibody is administered to a human patient according to a method of this invention. Other useful anti-CD4 antibodies are know to one of skill in the art and are useful according to the invention. In addition, derivatives of antibodies such as anti-CD4 small chain variable fragments (scFvs), minibodies, or diabodies directed against CD4 may be used according to the invention. Such molecules can be made according to techniques known in the art (see, e.g., Holliger et al., (2005), Nature Biotech., 23(9):1126-1136). Other anti-CD4 antibodies can be created according to methods known to one of skill in the art. In an alternate embodiment, an anti-CD4 antagonist includes any chemical moiety capable of binding to CD4.

It is an insight of this invention that, whereas T_{reg} cells remain functionally inactivated as a consequence of treating a mammal with the combination of an anti-CD25 antibody and an IL-2 protein composition, CD8+ cells and NK cells are expanded. Surprisingly, as is shown in Example 1 of this application, the effect on CD8+ cell and NK cell expansion is not seen with free (monomeric) recombinant IL-2, but is seen with other IL-2 protein compositions, such as an antibody-IL-2 fusion protein.

Without wishing to be bound by theory, it is possible that using multimeric forms of the IL-2 protein composition provide a sufficiently high local concentration of IL-2 to allow for the activation of the intermediate IL-2 receptor complex dependent signaling pathway. It appears that, in the presence of a blocking CD25 antagonist, such as an anti-CD25 antibody, certain T-cell subsets such as CD8+ memory T-cells or CD8+ effector T-cell are capable of responding to IL-2 signaling mediated by the intermediate-affinity IL-2 receptor complex, which does not contain CD25. However, T_{reg} cells, which are critically dependent on a high-affinity IL-2 receptor pathway for their activation by IL-2, do not respond to IL-2 when the receptor is blocked by the presence of a CD25 receptor antagonist, such as, for example, by an anti-CD25 antibody.

Accordingly, in another aspect, the invention is a method for the treatment of cancers. For example, in one embodiment, useful IL-2 protein compositions are antibody-IL2 fusion proteins which direct the IL-2 activity to the tumor microenvironment. Thus, according to a further embodiment, the fusion partner for IL-2 is an antibody moiety that has specificity for an antigen that is enriched in the tumor microenvironment. For example, antibody IL-2 fusion proteins where the antibody portion is the KS antibody, which recognizes the adhesion molecule EpCAM; the 14.18 antibody, which recognizes the disialoganglioside GD2; or the NHS76 antibody, which recognizes DNA in the necrotic core of tumors, are useful according to the invention. Other antibody moieties known to persons skilled in the art may be used, according to the cancer of the patient. IL-2 fusion proteins, according to one embodiment of the invention, include one or more mutations to the IL-2 portion of the fusion protein that reduce or abolish the interaction between IL-2 and the IL-2 high-affinity receptor α subunit. Useful mutations to IL-2 are described above.

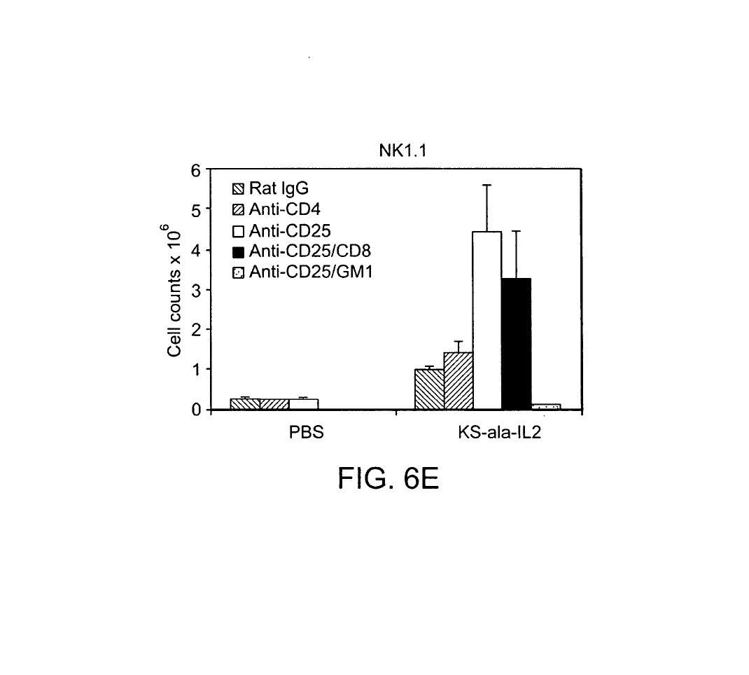

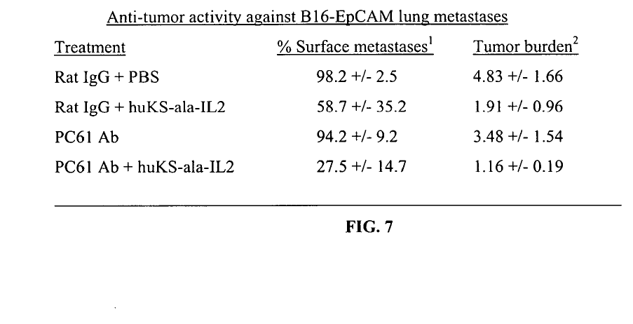

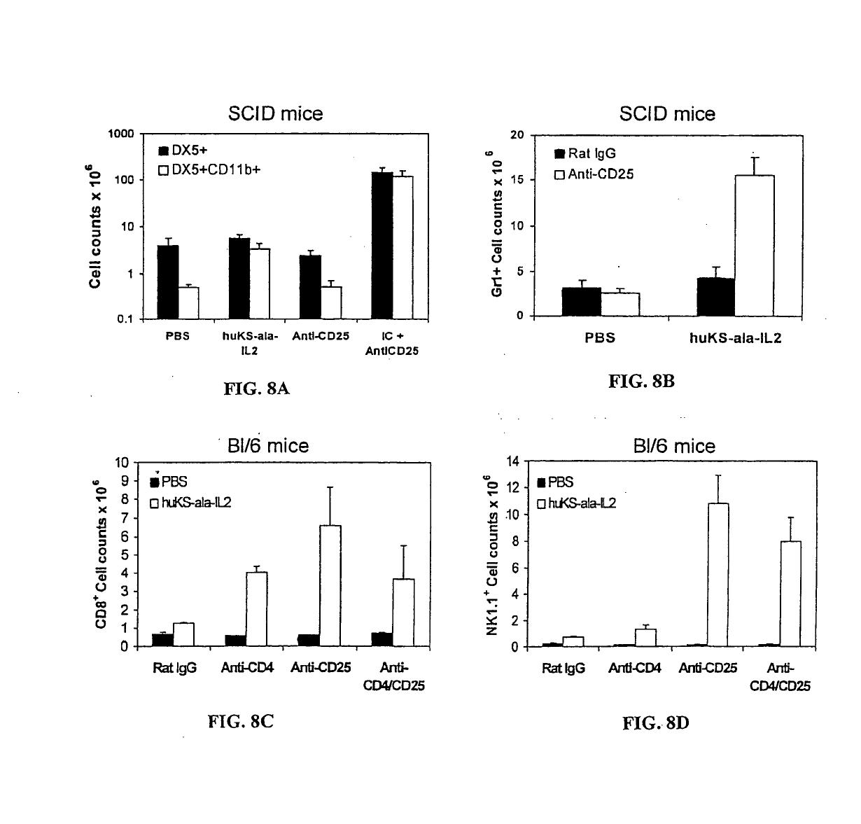

In a further embodiment, the antibody fusion protein is KS-IL2 (KS antibody with C-terminal heavy chain IL-2 moieties). Sequences for the light chain (SEQ ID NO:2) and heavy chain (SEQ ID NO:3) of the KS portion of KS-IL2 are shown in FIGS. 12 and 13, respectively. In another embodiment, the antibody fusion protein is KS-ala-IL2 (KS antibody with C-terminal heavy chain IL-2 moieties, with the C-terminal lysine of the antibody moiety substituted with alanine; also known as EMD 273066 or tucotuzumab celmoleukin; see also U.S. Pat. No. 5,650,150, and U.S. Patent Application Publication No. 2003/0157054). Sequences for the light chain (SEQ ID NO:2) and the heavy chain (SEQ ID NO:4) of KS-ala-IL2 are shown in FIGS. 12 and 14 respectively. (See also U.S. Patent Application Publication No. 2002/0147311).

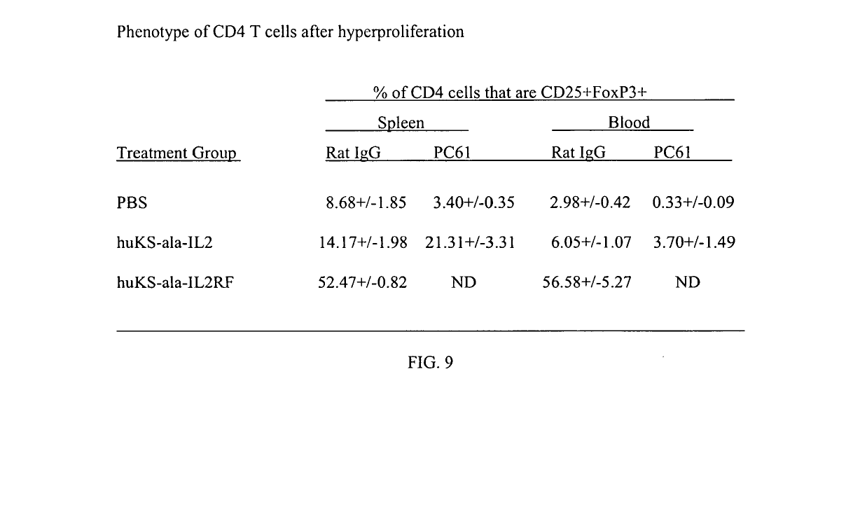

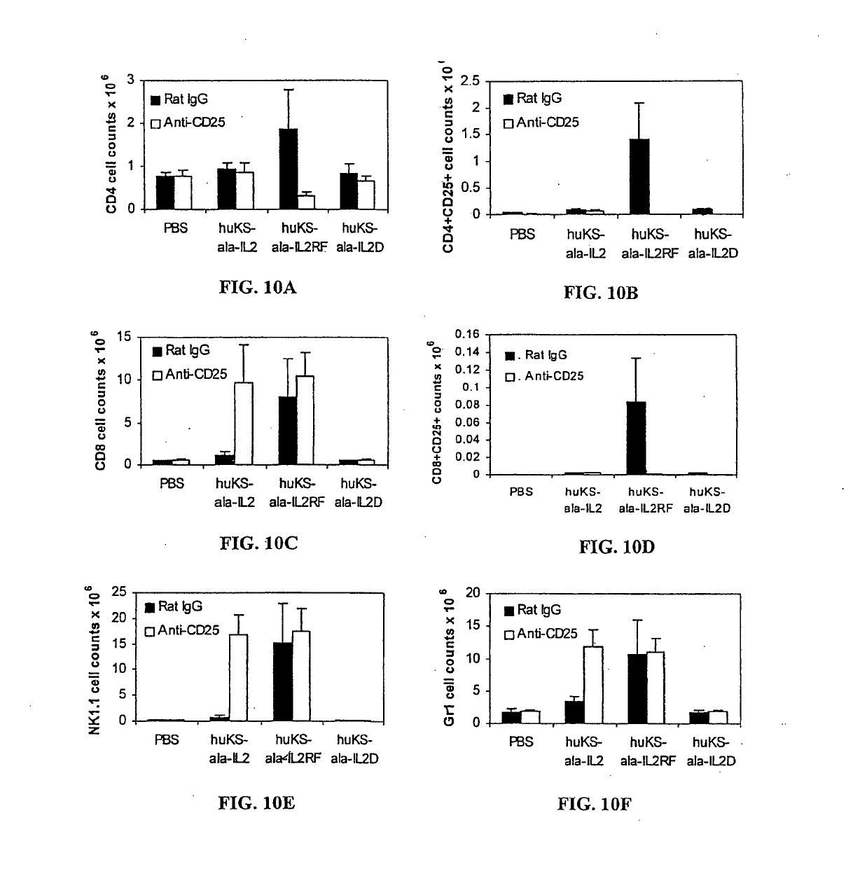

In a further embodiment, the antibody fusion protein is NHS76-IL2 (NI-IS 76 antibody with C-terminal heavy chain IL-2 moieties). Sequences for the light chain (SEQ ID NO: 5) and the heavy chain (SEQ ID NO:6) of an exemplary embodiment of NHS76-IL2 are shown in FIGS. 15 and 16 respectively. (See also U.S. Patent Application Publication No. 2002/0147311).

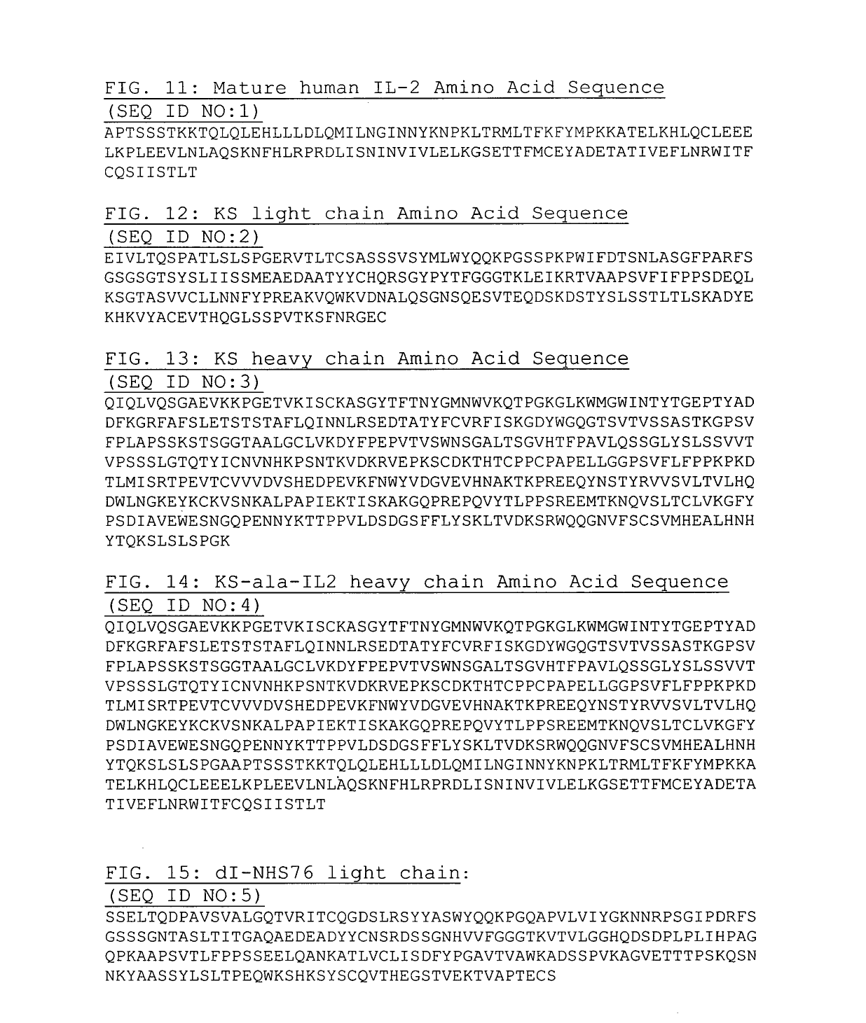

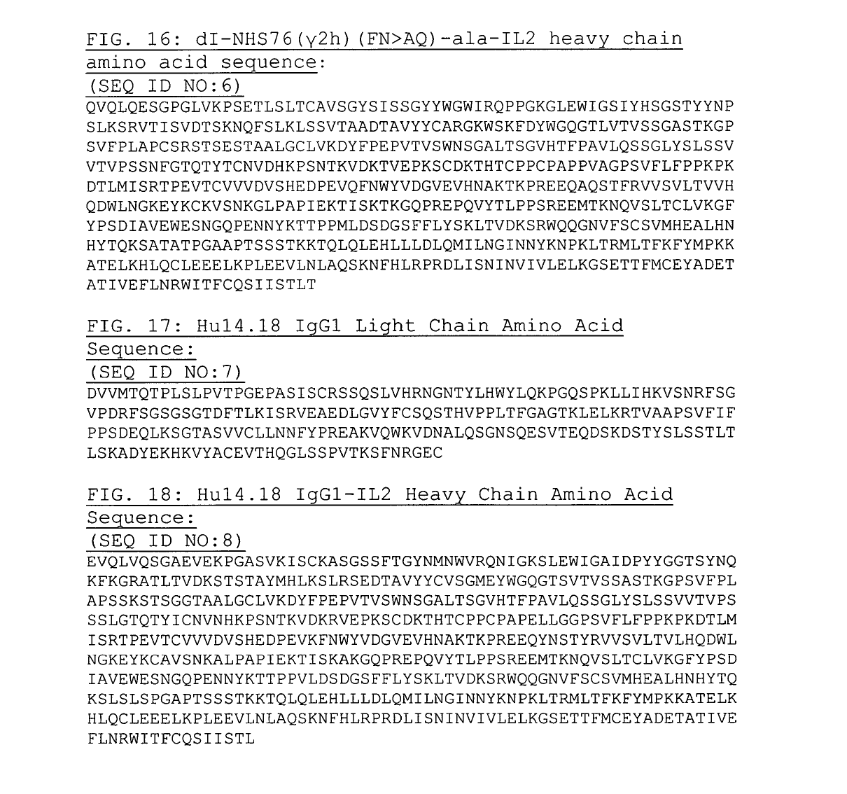

In a further embodiment, the antibody fusion protein is hu14.18-IL2 (human 14.18 antibody with C-terminal heavy chain IL-2 moieties). Sequences for the light chain (SEQ ID NO: 7) and heavy chain (SEQ ID NO:8) of an exemplary embodiment of hu14.18-IL2 are shown in FIGS. 17 and 18 respectively.

According to one embodiment of the invention, the IL-2 protein composition contains multiple copies of IL-2, i.e., is multimeric. For example, in one embodiment, the IL-2 protein composition contains two, three, four, five or more IL-2 moieties. Accordingly, in a further embodiment, the IL-2 protein composition is dimeric IL-2. According to a further embodiment, the IL-2 protein composition includes two IL-2 moieties joined to one another. According to the invention, the IL-2 moieties may be joined by a polypeptide linker, a chemical linker, a disulfide bond or the like. In a further embodiment, three, four, five or more IL-2 moieties are joined to form multimeric IL-2.

According to another embodiment of the invention, the multimeric IL-2 protein composition is an immunoglobulin fusion protein. For example, in one embodiment, the immunoglobulin fusion protein is an antibody-IL2 fusion protein. In another embodiment, an IL-2 moiety is joined to each heavy chain C-terminus of the antibody to form an antibody-IL2 fusion protein with two IL-2 moieties. In another embodiment, an IL-2 moiety is joined to each light chain N-terminus of an antibody to form an antibody-IL2 fusion protein with two IL-2 moieties. According to yet another embodiment, an antibody-IL2 fusion protein can include IL-2 moieties joined to one or more of the C-terminus and/or N-terminus of the heavy chain and/or the light chain to create a multimeric antibody-IL2 fusion protein. In a further embodiment, binding sites for FcγRs contained in the Fc region of the fusion protein are removed. (See U.S. Patent Application No. 2002/0105294). In a further embodiment, the immunoglobulin and IL-2 moieties are derived from a human, and therefore are useful in treating a human patient. According to one embodiment, the IL-2 moiety is joined to the antibody by fusion, i.e., incorporation into the protein backbone.

According to another embodiment of the invention, the multimeric IL-2 protein composition is an Fc-IL2 fusion protein. For example, in one embodiment, an IL-2 moiety is joined to each N-terminus of the Fc moiety to form an Fc fusion protein with two IL-2 moieties. In another embodiment, an IL-2 moiety is joined to each C-terminus of the Fc moiety to from an Fc fusion protein with two IL-2 moieties. According to a further embodiment, IL-2 moieties are joined to one or more of the N-terminus and/or C-terminus of the Fc moiety to create a multimeric Fc-IL2 fusion protein. According to one embodiment, the IL-2 moiety is joined to the Fc moiety by fusion, i.e., incorporation into the protein backbone. In a further embodiment, the immunoglobulin and IL-2 moieties are derived from a human, and therefore are useful in treating a human patient. Fusion proteins can be constructed according to standard procedures known to one of skill in the art, such as those procedures discussed in U.S. Pat. Nos. 5,650,150, 5,541,087, and 6,992,174 as well as U.S. Patent Application Publication Nos. 2002/0147311, 2003/0044423 and 2003/0166163.

According to the invention, in one embodiment, the IL-2 moiety includes one or more amino acid variants from wild-type IL-2. For example, in one embodiment, it is useful to mutate one or more of the following amino acid residues of the IL-2 moiety corresponding to the residues of the IL-2 wild type sequence shown in SEQ ID NO: 1: Lys8, Gln13, Glu15, Leu19, Asp20, Gln22, Met23, Asn26, Arg38, Phe42, Lys43, Thr51, His79, Leu80, Arg81, Asp84, Asn88, Val91, Ile92, and Glu95. According to another embodiment, it is also useful to mutate one or more of the following amino acid residues of the IL-2 moiety corresponding to the residues of the IL-2 wild-type sequence shown in SEQ ID NO: 1: Leu25, Asn31, Leu40, Met46, Lys48, Lys49, Asp109, Glu110, Ala112, Thr113, Val115, Glu116, Asn119, Arg120, Ile122, Thr123, Gln126, Ser127, Ser130, and Thr131.

In another embodiment according to the invention, the IL-2 moiety does not include a mutation that changes the affinity of the protein having an IL-2 moiety for the intermediate-affinity IL-2 receptor relative to the affinity for the intermediate-affinity IL-2 receptor of a protein having a wild-type IL-2 moiety. In yet another embodiment, the IL-2 moiety does not include a mutation that reduces the affinity of the protein having an IL-2 moiety for the intermediate-affinity IL-2 receptor relative to the affinity for the intermediate-affinity receptor of a protein having a wild-type IL-2 moiety.

In yet another embodiment according to the invention, the protein having an IL-2 moiety does not include a mutation that changes the protein having an IL-2 moiety's affinity for the high-affinity IL-2 receptor relative to the affinity of a protein having a wild-type IL-2 moiety's affinity for the high-affinity IL-2 receptor. In a further embodiment, the protein having an IL-2 moiety does not include a mutation that reduces the protein having an IL-2 moiety's activation of cells expressing the intermediate-affinity receptor relative to a protein having a wild-type IL-2 moiety's activation of cells expressing the intermediate-affinity receptor. In yet another embodiment, the protein having an IL-2 moiety does not have a mutation that reduces the protein having an IL-2 moiety's affinity for the intermediate-affinity IL-2 receptor relative to the affinity for the high-affinity IL-2 receptor. According to these embodiments, the protein having wild-type IL-2 moiety is a reference protein identical to the protein having an IL-2 moiety, except that the IL-2 moiety is a wild-type IL-2 moiety. Methods for comparing relative affinities of IL-2 containing proteins are discussed in U.S. Patent Application Publication No. 2003-00166163.

In another embodiment, the protein having an IL-2 moiety does not include a mutation that alters the protein having an IL-2 moiety's selectivity of the protein relative to the selectivity of a reference protein, the reference protein being identical to the protein having an IL-2 moiety, but that the IL-2 moiety of the reference protein is wild-type IL-2. The selectivity is measured as a ratio of activation of cells expressing the high-affinity IL-2 receptor relative to the activation of cells expressing the IL-2 intermediate-affinity receptor.

In another embodiment, the protein having an IL-2 moiety does not include a mutation that results in a differential effect on the protein having an IL-2 moiety's affinity for the IL-2 intermediate-affinity receptor relative to the protein having an IL-2 moiety's affinity for the IL-2 high-affinity receptor. The differential effect is measured by the proliferative response of cell or cell lines that depend on IL-2 for growth. This response to the protein having an IL-2 moiety is expressed as an ED50 value, which is obtained from plotting a dose response curve and determining the protein concentration that results in a half-maximal response. The ratio of the ED50 values obtained for cells expressing the intermediate-affinity IL-2 receptor for a protein having an IL-2 moiety relative to the ratio of ED50 values for a reference protein being identical to the protein having an IL-2 moiety, but wherein the IL-2 moiety is wild-type IL-2, gives a measure of the differential effect of the fusion protein.

According to the invention, in a further embodiment, the IL-2 moiety does not include a mutation at any of the following residues of the IL-2 moiety corresponding to the residues of the IL-2 wild-type sequence shown in SEQ ID NO:1: Lys8, Gln13, Glu15, Leu19, Asp20, Gln22, Met23, Asn26, Arg38, Phe42, Lys43, Thr51, His79, Leu80, Arg81, Asp84, Asn88, Val91, Ile92, and Glu95. According to another further embodiment of the invention, the IL-2 moiety does not include a mutation at any one of the following residues of the IL-2 moiety corresponding to the residues of the IL-2 wild-type sequence shown in SEQ ID NO:1: Leu25, Asn31, Leu40, Met46, Lys48, Lys49, Asp109, Glu110, Ala112, Thr113, Val115, Glu116, Asn119, Arg120, Ile122, Thr123, Gln126, Ser127, Ser130, and Thr131. A mutation, in one embodiment, is an insertion of an amino acid residue, while in another embodiment, a mutation is a deletion of an amino acid residue, while in yet another embodiment, a mutation is a substitution of an amino acid residue. In a further embodiment, the IL-2 moiety does not have a mutation at any one of the following residues corresponding to wild-type IL-2: D20T, N88R, or Q126D.

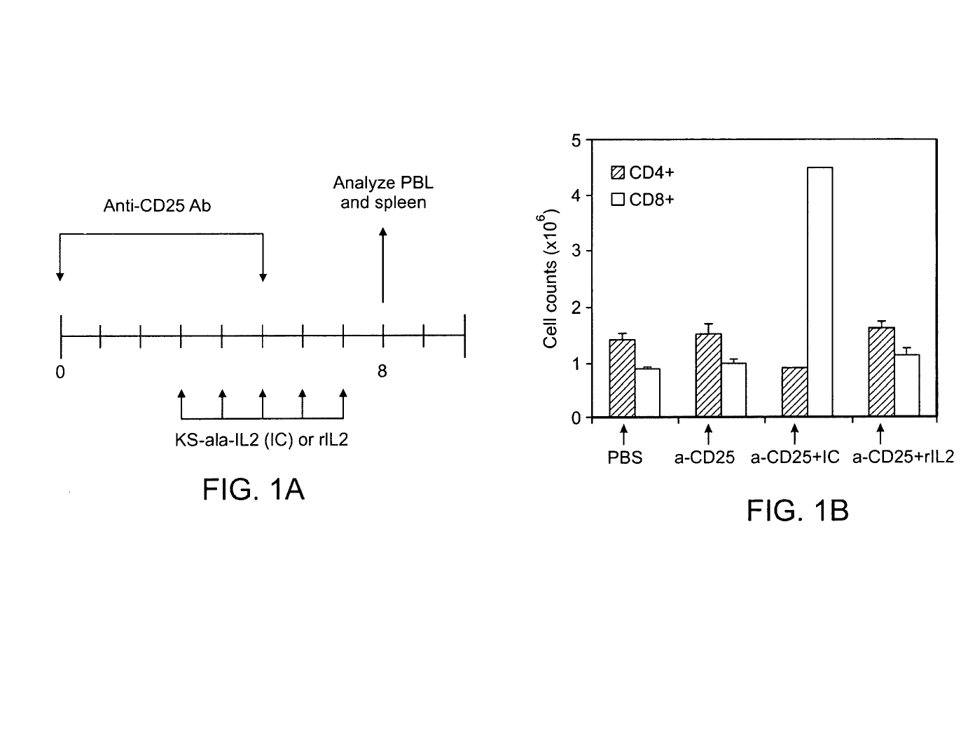

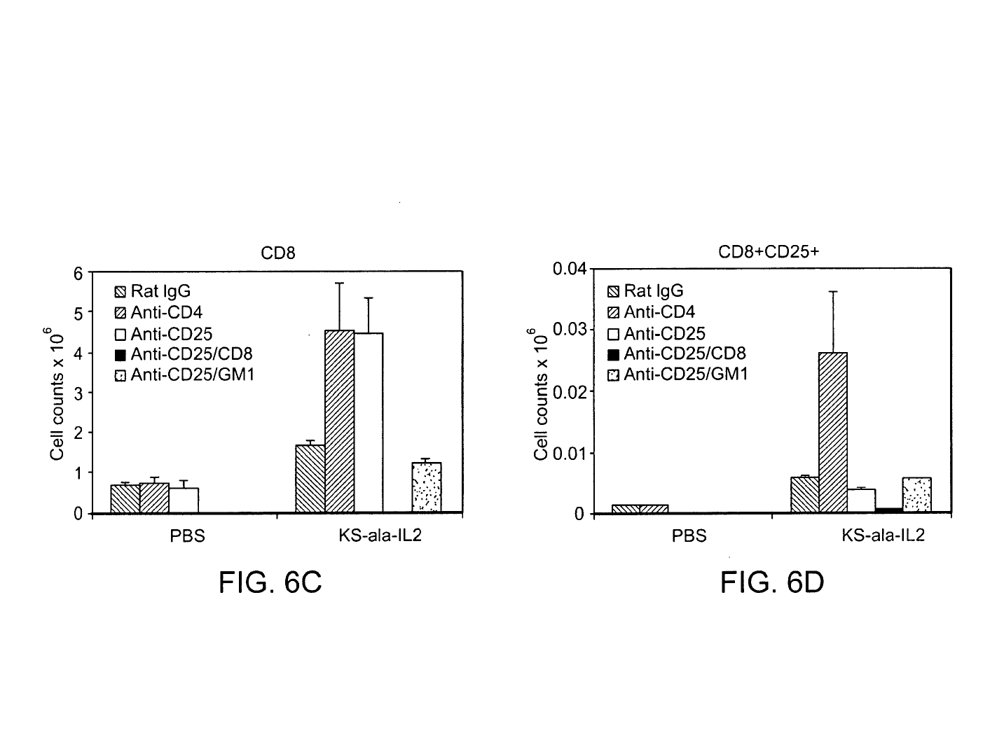

The invention contemplates not only using IL-2 sequences found in nature, such as the mature human wild-type IL-2 amino acid sequence disclosed in FIG. 11 (SEQ ID NO:1), but also contemplates using other IL-2 amino acid sequences that have, for example, at least 70%, at least 75%, at least 80%, at least 85%, at least 90%, at least 91%, at least 92%, at least 93%, at least 94%, at least 95%, at least 96%, at least 97%, at least 98%, or at least 99% amino acid identity with the mature human IL-2 amino acid sequence disclosed in FIG. 1.

To determine the percent identity of two amino acid sequences or to nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequence is a function of the number of identical positions shared by the sequences (i.e., % identity=# of identical positions/total # of positions×100).

The invention also contemplates using IL-2 sequences that maintain the biological activity of IL-2 of at least 10%, 20%, 30%, 40%, 50%, 60%, 70%, 80%, 90%, 92%, 95%, and even more preferably 99% as compared to mature human wild type IL-2 as shown in SEQ ID NO: 1. IL-2 activity can be measured using an in vitro cell proliferation assay, such as the assay described in U.S. Patent Application Publication No. 2003-0166163, or according to other methods known to one of skill in the art.

The invention also contemplates using multimeric IL-2 proteins. A multimeric IL-2 protein may be a protein composition comprising multiple polypeptide regions or moieties exhibiting IL-2 activity, linked together directly or indirectly by a peptide bond, a disulfide bond or a chemical linker. For instance, multimeric IL-2, in one embodiment, includes dimeric IL-2, which is a protein having two moieties each exhibiting IL-2 activity.

The term “CD25 receptor antagonist” means, in one embodiment, a polypeptide, nucleic acid or other chemical agent capable of binding to and disabling the CD25 subunit of the high-affinity IL-2 receptor. For example, in one embodiment, the CD25 receptor antagonist is an anti-CD25 antibody. According to another embodiment, the term “anti-CD25 antibodies” includes all anti-CD25 antibodies that are CD25 receptor antagonists. CD25 antagonists include, for example, antagonists that cause degradation of the CD25 subunit, antagonists that cause internalization of the CD25 subunit, antagonists that block IL-2 binding to the CD25 subunit, or antagonists that cause interference with the interaction of the CD25 subunit with the other subunits of the high-affinity IL-2 receptor.

In another embodiment, the term “CD25 receptor antagonist” also includes other polypeptides, nucleic acids, or other chemical agents capable of binding to IL-2, thereby interfering with IL-2's ability to bind to the α subunit (CD25) of the high-affinity IL-2 receptor. Chemical agents capable of interfering with IL-2's ability to bind the α subunit are discussed in Rickert et al., (2005), Science, 308:1477-1480. For example, in one embodiment, the CD25 antagonist is an anti-IL-2 antibody directed against at least a portion of the IL-2 moiety necessary for binding to the α subunit (CD25) of the high-affinity IL-2 receptor. Examples of anti-IL-2 antibodies directed against at least a portion of the IL-2 moiety necessary for binding to the α subunit (CD25) of the high-affinity IL-2 receptor are known in the art and include the murine monoclonal antibody S4B6 and the monoclonal antibody MAB602 directed against human IL-2, disclosed in Boyman et al., Science, (2006), 311:1924-1927. According to another embodiment of the invention, a CD25 receptor antagonist, as defined herein, does not block the interaction between IL-2 and the β receptor of an IL-2 receptor, such as is present in the intermediate-affinity or high-affinity IL-2 receptor

According to one embodiment, an “antibody” means an intact antibody (for example, a monoclonal or polyclonal antibody. According to another embodiment, an antibody may include antigen binding portions thereof, including, for example, an Fab fragment, an Fab′ fragment, an (Fab′)₂ fragment, an Fv fragment, a single chain antibody binding site, and an sFv, bi-specific antibodies and antigen binding portions thereof, and multi-specific antibodies and antigen binding portions thereof. Furthermore, in yet another embodiment, an antibody may encompass any of an Fab fragment, an Fab′ fragment, an (Fab′)₂ fragment, an Fv fragment, a single chain antibody binding site, or an sFv fragment linked to an Fc moiety or any portion of an Fc moiety.

According to the invention, an “anti-CD25” antibody in one embodiment is an antibody capable of specific binding to the CD25 subunit (antigen) of the IL-2 high-affinity receptor. “Specific binding,” “bind specifically,” and “specifically bind” are understood to mean that the antibody has a binding affinity for the antigen of interest of at least about 10⁻⁶ M, alternately at least about 10⁻⁷M, alternately at least about 10⁻⁸M, alternately at least 10⁻⁹M or alternately at least about 10⁻¹⁰ M.

According to one embodiment of the invention, the method of treatment affects the balance of T_{reg} cells and activated CD8+ effector cells in favor of CD8+ effector cells. In one embodiment of the invention, an anti-CD25 antibody is used that functionally inhibits IL-2 dependent signaling in cells expressing the high-affinity IL-2 receptor complex. For example, anti-CD25 antibodies are used that, like PC61 or 7D4, are shown to lead to the functional inactivation of T_{reg} cells (Kohm et al., (2006), J. Immunol., 176:3301-3305). In a further embodiment, the anti-CD25 antibody optionally may include mutations that reduce its circulating half-life. Methods to obtain such antibodies are known in the art. For example, an antibody with a deletion of the CH2 domain is used. Alternatively, in another embodiment, an antibody with reduced binding to the FcRn receptor is used, such as with a point mutation at His435. Such antibody embodiments may be useful in favoring the expansion of CD8+ effector T-cells over T_{reg} cells upon stimulation with an IL-2 protein composition. Moreover, such antibody embodiments may be useful in conjunction with IL-2 protein compositions that signal through the high-affinity IL-2 receptor complex and not the intermediate-affinity IL-2 receptor complex.

In another embodiment of the invention, anti-CD25 antibodies are used that lead to the depletion of T_{reg} cells. For example, anti-CD25 antibodies are used that elicit a strong CDC response or a strong ADCC response. Methods to increase CDC or ADCC are known in the art. For example, CDC response may be increased with mutations in the antibody that increase the affinity of C1q binding (Idusogie et al., (2001), J. Immunol., 166(4):2571-2575). ADCC may be increased by methods that eliminate the fucose moiety from the antibody glycan, such as by production of the antibody in a YB2/0 cell line. In another embodiment of the invention, anti-CD25 antibody conjugates with radionuclides or toxins are used. Commonly used radionuclides are, for example, ⁹⁰Y, ¹³¹I, and ⁶⁷Cu, among others, and commonly used toxins are doxirubicin, calicheamicin, or the maytansines DM1 and DM4 (Wu et al., (2005), Nat. Biotechnol., 23(9):1137-1146). In a further embodiment, the anti-CD25 antibody conjugates optionally may include mutations that reduces its circulating half-life. Methods to obtain such antibodies are known in the art. For example, an antibody with a deletion of the CH2 domain is used. Alternatively, an antibody with reduced binding to the FcRn receptor is used, such as with a point mutation at His435.

Antagonists of the CD25 receptor that are not based on antibodies may also be used. Such antagonists may be based, for example, on nucleic acid oligonucleotides, on peptides or on non-antibody polypeptide domains. In one embodiment of the invention, an antagonistic DNA aptamer against CD25 is used. In another embodiment of the invention, an antagonistic RNA aptamer against CD25 is used. Methods to obtain DNA and RNA aptamers are known in the art. The methods rely on an in-vitro iterative process of selecting nucleic acid molecules that bind the target protein and of amplifying the bound molecules, commonly referred to as SELEX (see, for example Brody et al., (2000) J Biotechnol 74:5-13). In a further embodiment, the anti-CD25 aptamer may additionally include modifications to enhance its therapeutic effectiveness. For example, nucleic acid analogs are introduced to render the aptamer resistant to nucleases or it may be conjugated to carrier molecules to enhance its circulating half-life. In another embodiment, the CD25 antagonist is derived from non-antibody polypeptide domains. Useful non-antibody polypeptide domains are known in the art and generally feature a scaffold structure onto which variable, potential epitope-binding, loops are engineered. For example, fibronectin Type III domains are used. Methods to obtain a CD25 antagonist based on a fibronectin scaffold are known in the art. For example, phage display technology, displaying a library of fibronectins with randomized surface loops, can be used to select for specific CD25 binders (see, e.g., U.S. Pat. No. 5,223,409). Alternatively, an in-vitro iterative selection technology is used (see, e.g., 6,818,418). Alternative methods for obtaining specific CD25 antagonists are, for example, designed with ankyrin repeat protein libraries (Binz et al., (2004) Nat Biotechnol 22(5):575-582), or with avimers (Silverman et al., (2005) Nat Biotechnol 23(12):1556-1561).

As used herein, the term “immunoglobulin” is understood to mean a naturally occurring or synthetically produced polypeptide, such as a recombinant polypeptide, homologous to an intact antibody (for example, a monoclonal or a polyclonal antibody) or a fragment or portion thereof, such as an antigen binding portion. Immunoglobulins according to the invention may be from any class such as IgA, IgD, IgG, IgE or IgM. IgG immunoglobulins can be of any subclass such as IgG1, IgG2, IgG3, or IgG4. The term immunoglobulin encompasses polypeptides and fragments thereof derived from immunoglobulins.

The constant region of an immunoglobulin is a naturally-occurring or synthetically-produced polypeptide homologous to the immunoglobulin C-terminal region, and can include a CH1 domain, a hinge, a CH2 domain, a CH3 domain, or a CH4 domain, separately or in any combination. As used herein, “Fc moiety” encompasses the hinge, the CH2 domain, the CH3 domain, or the CH4 domain derived from the constant region of an antibody, including a fragment, analog, variant, mutant, or derivative of the constant region, separately or in any combination. In one embodiment, the Fc moiety includes the hinge, CH2 domain and CH3 domain. Alternately, the Fc moiety, in another embodiment, includes all or a portion of the hinge, the CH2 domain and/or the CH3 domain.

According to the invention, constant domains of an antibody, in one embodiment, are derived from different IgG classes. For example, in one embodiment, the hinge region of an antibody is from IgG1, while the CH2 domain and CH3 domain are from IgG2. In a further embodiment, the hinge of an Fc moiety is from IgG1, while the CH2 domain and CH3 domain are from IgG2.

According to the invention, in one embodiment, the IL-2 protein composition includes an immunoglobulin moiety. According to a further embodiment of the invention, the immunoglobulin moiety does not include an alteration or mutation which affects the binding properties of the IL-2 protein composition to the IL-2 intermediate or high-affinity receptor. For example, in one embodiment, the immunoglobulin moiety does not include a modification that affects the glycosylation pattern of the protein. In another embodiment, the immunoglobulin moiety does not include a modification at position N297 of an IgG heavy chain. Modifications include PEGylation of the molecule and treatment with N-glycanase to remove N-linked glycosyl chains. In another embodiment, the immunoglobulin moiety does not include a mutation that directly affects interaction with an Fc receptor. In another embodiment, the immunoglobulin region does not have a mutation substituting another amino acid in place of the C-terminal lysine of the heavy chain. In a further embodiment, the C-terminal lysine is not substituted with alanine. In a further embodiment, the immunoglobulin moiety does not have a mutation that eliminates or reduces T-cell epitopes.

According to a method of the invention, a CD25 receptor antagonist, such as an anti-CD25 antibody is administered in conjunction with an IL-2 protein composition. In one embodiment, the anti-CD25 antibody and the IL-2 protein composition are administered apart from one another. In another embodiment, the anti-CD25 antibody is administered substantially simultaneously with the IL-2 protein composition. In one embodiment, pretreatment occurs with an anti-CD25 antibody, followed by an IL-2 protein composition.

According to one embodiment of the invention, the doses of the anti-CD25 antibody and of the IL-2 protein composition is administered together, while in another embodiment, the doses are administered separately during the same treatment session. In an alternate embodiment, the doses are administered during separate treatment sessions. For example, in a particular embodiment, a dose of anti-CD25 antibody is given on day 0, and a dose of the IL-2 protein composition is given zero to seven days later. In another particular embodiment, a dose of anti-CD25 antibody is given on day 0 and the dose of IL-2 protein composition is given on day 3. Other spacing regimens between the administrations may be used, as appropriate. In one embodiment, for example, spacing regimens are used under which the anti-CD25 antibody is effective against target cells such as T_{reg} cells, precluding the IL-2 protein composition from significantly simulating T_{reg} cells.

Optionally, according to another embodiment, a second dose of the anti-CD25 antibody is given. The intent is to achieve a sustained level of CD25 saturation. For example, in one embodiment, a second dose of anti-CD25 antibody may be given on day 5. It may be convenient to administer the second dose of anti-CD25 antibody on the same day as a second dose of an IL-2 protein composition, where the dosing regimen is determined by the optimal dosing regimen observed for multiple dosings of the IL-2 protein composition.

According to a method of the invention, a CD25 receptor antagonist, such as an anti-IL-2 antibody is administered in conjunction with an IL-2 protein composition. In one embodiment, the anti-IL-2 antibody and the IL-2 protein composition are administered apart from one another. In another embodiment, the anti-IL-2 antibody is administered substantially simultaneously with the IL-2 protein composition. In one embodiment, pretreatment occurs with an anti-IL-2 antibody, followed by an IL-2 protein composition.

According to one embodiment of the invention, the doses of the anti-IL-2 antibody and of the IL-2 protein composition are administered together, while in another embodiment, the doses are administered separately during the same treatment session. In an alternate embodiment, the doses are administered during separate treatment sessions. For example, in a particular embodiment, a dose of anti-IL-2 antibody is given on day 0, and a dose of the IL-2 protein composition is given zero to seven days later. In another particular embodiment, a dose of anti-IL-2 antibody is given on day 0 and the dose of IL-2 protein composition is given on day 3. Other spacing regimens between the administrations may be used, as appropriate. In one embodiment, for example, spacing regimens are used under which the anti-IL-2 antibody is effective against target cells such as T_{reg} cells, precluding the IL-2 protein composition from significantly simulating T_{reg} cells.

Optionally, according to another embodiment, a second dose of the anti-IL-2 antibody is given. The intent is to achieve a sustained level of IL-2 saturation by the anti-IL-2 antibody. For example, in one embodiment, a second dose of IL-2 antibody may be given on day 5. It may be convenient to administer the second dose of anti-IL-2 antibody on the same day as a second dose of an IL-2 protein composition, where the dosing regimen is determined by the optimal dosing regimen observed for multiple dosings of the IL-2 protein composition.

In a further embodiment, an IL-2 fusion protein having a mutation in the IL-2 moiety that reduces or eliminates the interaction between the IL-2 moiety and the α subunit of the high-affinity IL-2 receptor is administered to a patient on day zero. Thereafter, zero to seven days later, another dose of the mutant IL-2 fusion protein is administered. Other spacing regimens may be used as appropriate.

As is illustrated in Example 2 of this application using a pre-clinical mouse model, the method of this invention, according to one embodiment, was more effective when two successive doses of the IL-2 protein composition were used. For example, according to one embodiment, in a dosing regime that includes two doses of an IL-2 protein composition two days apart, the anti-CD25 antibody is administered on day 0 and day 5, and the IL-2 protein composition is administered on day 3 and day 5. This dosing regimen is illustrative of one embodiment of the invention; however, persons skilled in the art will recognize that variations of the dosing regimen may be contemplated without deviating from the spirit of the invention.

According to another embodiment, other treatments are optionally included to promote the activation of the immune system or the generation of CD8+ effector cells. For example, according to one embodiment, optional initial treatments with an IL-2 protein composition are included one to 14 days, preferably one to seven days, prior to the combination treatment described in the preceding paragraphs. According to a further embodiment of the invention, examples of other cytokines that may optionally be administered prior to the combination treatment of IL-2 and the CD25 antagonist are, for example, IL-7, IL-12, and/or IL-15. The method of the invention also contemplates the use of other immune system activating agents, such as the adjuvant CpG and others known to persons skilled in the art.

According to one embodiment of the invention, an anti-CD25 antibody is given at a dose at which a sustained saturation of CD25 receptors can be achieved. For example, to treat a human adult, a dose between about 0.01 mg/kg and 10 mg/kg is generally administered. In another embodiment, a dose between about 0.5 mg/kg and 2 mg/kg is used. In a particular embodiment, the anti-CD25 antibody daclizumab is standardly administered at about 1 mg/kg, intravenously, in a volume of 50 ml of a sterile 0.9% saline solution. In an alternate embodiment, an anti-CD4 antibody is administered instead of an anti-CD25 antibody according to any of the preceding protocols.

According to one embodiment of the invention, a fusion protein having a mutant IL-2 moiety is administered at a dose generally between about 0.01 mg/kg and 10 mg/kg. In another embodiment, a dose between about 0.5 mg/kg and 2 mg/kg is used. In a particular embodiment, the fusion protein having a mutant IL-2 moiety is administered at about 1 mg/kg, intravenously, in a volume of 50 ml of a sterile 0.9% saline solution.

According to one embodiment of the invention, an anti-IL-2 antibody is given at a dose to saturate the portion of the IL-2 moiety necessary for binding to the α subunit (CD25) of the high-affinity IL-2 receptor with anti-IL-2 antibody for a long period of time. For example, to treat a human adult, a dose between about 0.01 mg/kg and 10 mg/kg is generally administered. In another embodiment, a dose between about 0.5 mg/kg and 2 mg/kg is used. In a particular embodiment, the anti-IL-2 antibody is administered at about 1 mg/kg, intravenously, in a volume of 50 ml of a sterile 0.9% saline solution.

According to another embodiment of the invention, an IL-2 protein composition is also administered, at a dose determined to be below the maximal tolerated dose. In one embodiment, for antibody-IL2 fusion proteins or Fc-IL2 fusion proteins, a dose between about 0.004 mg/m² and 4 mg/m² is administered. In a further embodiment, a dose between about 0.12 mg/m² and 4 mg/m² is used. In a further embodiment, a dose between about 0.12 mg/m² and 1.2 mg/m² is used. In an even further embodiment, a dose of about 1 mg/m² is used, being administered intravenously in a 4 hour infusion. In another specific embodiment, a lower dose than standard is used, such as about 0.5 mg/m², as the method of the invention may provide a better therapeutic index for the antibody-IL2 fusion protein than a method in which the antibody-IL2 fusion protein is administered in isolation.

The combination therapy of the invention, using an antibody-IL-2 fusion protein, may be administered in the ways described in the preceding paragraphs.

According to another embodiment of the invention, the CD25 antagonist and the IL-2 protein composition are administered either parenterally, e.g., intravenously, intradermally, subcutaneously, orally (e.g., by inhalation), intraperitoneally, transdermally (topically), transmucosally, or rectally.

In another aspect of the invention, a method is provided that is more effective than a cancer vaccine alone in stimulating an immune response against a tumor. According to the invention, in one embodiment, the method can be used in conjunction with any desired cancer vaccine preparation. In general, cancer vaccines are directed against antigens expressed preferentially by tumor cells or by cells of the surround tumor stroma which support tumor growth. Examples of tumor-selective antigens include members of the MAGE family, members of the Cancer/Testis antigen family, survivin, CEA, or mucin, among others. Examples of antigens selective for cells of the tumor stroma are VEGFR1 or FAPaplha, among others. In other embodiments, the method is used to improve the efficacy for another antigen of interest.

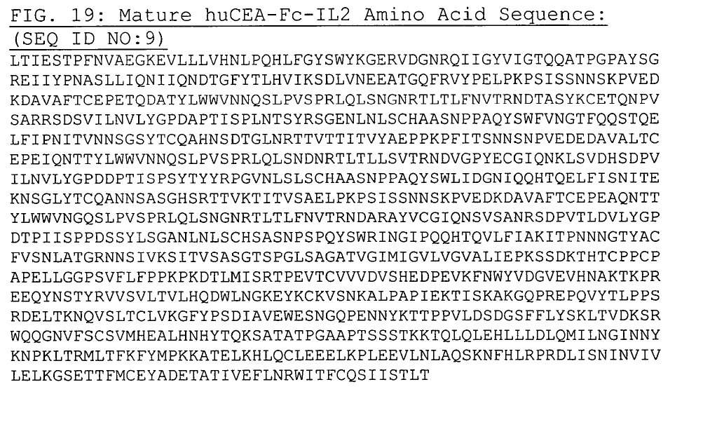

Cancer vaccine compositions may be based on DNA encoding the antigenic entity or on polypeptides that may form the antigenic precursor or the antigenic entity itself. DNA-based cancer vaccine compositions may be delivered either as naked DNA, or in a delivery vehicle, such as a liposome, or a virus or a bacterium. For example, in one embodiment, a DNA vaccine encoding survivin may be used, packaged for delivery in a salmonella-based bacterial vehicle as described, for example, by Xiang et al. in U.S. Patent Application Publication No. 2004/0192631. In a further embodiment, a polypeptide-based cancer vaccine composition is used, comprising a cocktail of peptides. For example, in one embodiment, a cocktail of peptides including ones described by Straten et al. in U.S. Patent Application Publication No. 2004/0210035 is used. In yet a further embodiment, a protein such as a CEA-Fc-IL2 is used (SEQ ID NO: 9, see, e.g., FIG. 19), with CEA being the antigen and the Fc-IL2 moiety having an adjuvant effect and additionally providing IL-2 activity.

In one embodiment of the invention, the cancer vaccine protocol is used in conjunction with a combination therapy that includes an anti-CD25 antibody and a protein composition containing multiple copies of IL-2. For example, in one embodiment, pretreatment with a cancer vaccine is followed with treatment by an IL-2 protein composition and an anti-CD25 antibody. In one embodiment, the IL-2 protein composition is administered first after pretreatment with the vaccine, and is followed by administration of an anti-CD25 antibody. In another embodiment, the anti-CD25 antibody and IL-2 protein composition are administered together after pretreatment with the vaccine. Examples of treatment regimens are outlined in Example 11 and Example 12, and in the preceding paragraphs. In a different embodiment of the invention, an anti-IL-2 antibody is substituted for the anti-CD25 antibody.

In one embodiment, a method of enhancing the efficacy of a vaccine includes administering to a patient an antigen of the vaccine and an IL-2 fusion protein containing one or more mutations that reduce or abolish the interaction between IL-2 and the α subunit of the high-affinity IL-2 receptor. Useful mutations to IL-2 are discussed above. In another embodiment, the method includes administering to a patient an antigen of a vaccine and a nucleic acid encoding an IL-2 fusion protein containing one or more mutations that reduce or abolish the interaction between IL-2 and the α subunit of the IL-2 receptor. According to a further embodiment, one vaccine according to the invention is a pharmaceutical composition comprising an antigen and an IL-2 fusion protein containing one or more mutations that reduce or abolish the interaction between IL-2 and the α subunit of the IL-2 receptor.

According to another embodiment of the invention, a method of enhancing the efficacy of a vaccine includes administering to a patient an antigen of the vaccine, an IL-2 fusion protein, and a protein that binds IL-2. In one embodiment, the IL-2 fusion protein is an antibody-IL-2 fusion protein. In a further embodiment, the method includes administering to a patient a vaccine, a protein that binds IL-2, and a nucleic acid encoding an IL-2 fusion protein. According to a further embodiment, one vaccine according to the invention is a pharmaceutical composition comprising an antigen, an IL-2 fusion protein, and a protein that binds IL-2.

According to another embodiment of the invention, a method of enhancing the efficacy of a vaccine includes administering to a patient a vaccine, an IL-2 fusion protein, and an inhibitor of the interaction between IL-2 and an IL-2 receptor α subunit. In one embodiment, the IL-2 fusion protein is an antibody-IL-2 fusion protein. In a further embodiment, the method includes administering to a patient an antigen of a vaccine, an inhibitor of the interaction between IL-2 and an IL-2 receptor α subunit, and a nucleic acid encoding an IL-2 fusion protein. According to another embodiment, one vaccine according to the invention is a pharmaceutical composition comprising an antigen, an IL-2 fusion protein, and an inhibitor of the interaction between IL-2 and an IL-2 receptor α subunit. In one embodiment, the vaccine is administered to a patient.

In another aspect, the invention includes a pharmaceutical composition comprising an IL-2 protein and a protein that blocks the interaction between IL-2 and the IL-2 α subunit of the high-affinity IL-2 receptor. For example, in one embodiment, the pharmaceutical composition includes IL-2 and an anti-CD25 antibody. In one embodiment, the pharmaceutical composition is a mixture, such as a solution, of IL-2 and anti-CD25 antibodies. In another embodiment, the pharmaceutical composition includes IL-2 and an anti-IL-2 antibody. For example, the pharmaceutical composition can be a mixture, such as a solution of IL-2 and anti-IL-2 antibodies. In a further embodiment, the IL-2 is an IL-2 fusion protein. In a further embodiment, the IL-2 fusion protein does not block the interaction between IL-2 and the IL-2 intermediate-affinity or high-affinity receptor β subunit. In yet a further embodiment, the IL-2 fusion protein does not block the interaction between IL-2 and the IL-2 high-affinity receptor α subunit. In another embodiment, the IL-2 protein includes a mutation that reduces or eliminates the ability of IL-2 to bind to the α subunit of the high-affinity IL-2 receptor. In a further embodiment, the pharmaceutical composition is administered to a patient, for example, a human patient.

In another aspect, the invention includes kits. According to the invention, a kit, in one embodiment, is used in a method for stimulating effector cell function in a patient. In another embodiment, the kit is used in a method for modulating IL-2 mediated immune response. The kit, according to one embodiment, includes at least a CD25 receptor antagonist and an IL-2 protein composition. In one embodiment, the CD25 receptor antagonist is contained in one container and the IL-2 protein composition is contained in another container within the kit. In yet another embodiment, the CD25 receptor antagonist is contained in the same container as the IL-2.

With continued reference to kits encompassed by the invention, in one embodiment, the IL-2 contained in the kit is mutated to reduce or eliminate the ability of IL-2 to bind to the CD25 subunit of the IL-2 high-affinity receptor. For example, in one embodiment, IL-2 has mutations at one or more residues corresponding to R38W and F42K. In one embodiment, the CD25 receptor antagonist is an anti-CD25 antibody, while in another embodiment, the CD25 receptor antagonist is an anti-IL-2 antibody. In a further embodiment, the anti-IL-2 antibody is directed against at least a portion of the IL-2 moiety necessary for binding to the α subunit (CD25) of the high-affinity IL-2 receptor of IL-2.

In a further embodiment, the IL-2 contained within the kit according to the invention is an IL-2 fusion protein.

The invention is further illustrated by the following non-limiting Examples.

### Example 1

### Enhancement of CD8+ Cells in Mice Treated with an Anti-CD25 Antibody and an Antibody-IL2 Fusion Protein

To assess in a mouse model the effect of the combination therapy of an anti-CD25 antibody with various forms of IL-2, the changes in the level of mouse immune cells collected from peripheral blood and the spleen after treatment were analyzed.

Seven to eight week old female C57BL/6 mice were used. Mice (n=3 per treatment group) were administered intraperitoneally with the rat anti-mouse anti-CD25 antibody PC61 (produced from rat hybridoma cells PC61, ATCC TIB222, Manassas, Va.) at a dose of 250 micrograms/mouse on day 0 and day 5. Mice in one experimental group were treated further with the antibody fusion protein KS-ala-IL2 intravenously at a daily dose of 20 μg/mouse from day 3 to day 7, whereas mice in a second experimental group were treated further with recombinant human IL-2 (rh-IL2) intravenously at a daily dose of 3.3 μg/mouse, which, on a molar basis, provided the equivalent dose with respect to IL-2 to the mouse as 20 micrograms/mouse of KS-ala-IL2 did (see FIG. 1A). In control groups, mice received only the PC61 antibody treatment as above, either at a dose of 250 micrograms/mouse or 100 micrograms/mouse, or injections of 0.2 ml of PBS solution.

Peripheral blood cells were collected at the start of IL-2 treatment on day 3, and again at the conclusion of IL-2 treatment on day 8, and analyzed by standard techniques of flow cytometry, familiar to those skilled in the art, for the markers CD4, CD8 and CD25. Spleens were harvested on day 8 and analyzed as above. At day 3, while the number of total CD4+ and total CD8+ cells remained relatively unchanged the number of detectable CD25+ cells decreased to approximately 10% relative to the PBS-treated control group, confirming previously reported effects of this antibody. Moreover, a comparison of the two doses of the PC61 antibody showed that the lower dose of 100 μg was as effective as the higher dose at reducing the number of detectable CD25+ cells and therefore was typically the dose used in subsequent experiments. At day 8, peripheral blood cells from mice treated with the combination of PC61 and KS-ala-IL2 (SEQ ID NOS: 2 and 4) showed dramatic changes in the CD4+ and CD8+ cell populations relative to the PBS-treated controls or mice treated only with PC61: total CD4+ cells decreased by nearly 40% while total CD8+ cells increased by more than 400%. Thus, the combination treatment had opposing effects on total CD4+ and CD8+ populations.

Surprisingly, in contrast to the effect of KS-ala-IL2, peripheral blood cells from mice treated with the combination of PC61 and rhIL-2 showed no significant changes in these cell populations and the numbers were similar to the controls (FIG. 1B). A similar result was seen with cells isolated from spleen (FIG. 1C). Furthermore, analysis of the spleen cells with respect to CD25+ cells showed that PC61 antibody treatment was effective and its effect persisted for the duration of the experiment, reducing the number of detectable CD25+ cells, including CD4+CD25+ cells, to less than 10% of control (FIG. 1D).

These results indicate that, when combined with an antibody to CD25, such as PC61, treatment of an animal with IL-2 in the context of an antibody-IL2 fusion protein, in contrast to treatment with free (monomeric) IL-2, leads to significant and beneficial alterations in immune cell populations useful for immunotherapy, namely a boost in the number of CD8+ cells and a reduction in the number of detectable CD25+ cells, including CD4+CD25+ cells, a T_{reg} cell population. Free IL-2 does not produce the same beneficial results as the antibody-IL2 fusion protein.

Without wishing to be bound by theory, the differential effect seen with KS-ala-IL2 relative to free IL-2 (SEQ ID NO:1) may be due to increased local concentration of IL-2 moieties, such as by an avidity effect, at the relevant cells with KS-ala-IL2. It suggests that other compositions that provide an avidity effect for IL-2, such as other protein variants containing dimeric IL-2 proteins, for example an Fc-IL2 or antibody-IL2 fusion protein, may be effective in combination with an anti-CD25 antibody.

It has been reported that, in mice, T_{reg} cells are not physically depleted by anti-CD25 antibody treatment, but rather, the CD25 receptor protein on T_{reg} cells is down-regulated or shed, leading to a functional inactivation of T_{reg} cells (Kohm et al., (2006), J. Immunol., 176:3301-3305). This observation is consistent with results from a separate experiment performed essentially as described above, but in addition using a reagent to detect cells expressing the transcription factor FoxP3, which in conjunction with CD4 is characteristic of T_{reg} cells. It was found that although CD4+CD25+ cells were not detectable after treatment with PC61 antibody, CD4+FoxP3+ cells were detectable, indicating that T_{reg} cells were not depleted and continued to express FoxP3, but rather functionally inactivated with respect to CD25-dependent stimulation (data not shown). Without wishing to be bound by theory, it is likely that the IL-2 activity provided by the dimeric nature of IL-2 in the context of an antibody-IL2 fusion protein overcomes what could be expected to be an inhibitory effect by the anti-CD25 antibody to expand the CD8+ cell population, but not the CD4+CD25+ cell population.

Interestingly, the combination therapy led to a diminution rather than to an expansion of total CD4+ cells, further suggesting that CD4+ and CD8+ cells do indeed respond differently to the treatment. Without wishing to be bound by theory, it is likely that T_{reg} cells, being a type of CD4+ cell, are also not responsive to antibody-IL2 fusion protein treatment in the context of the combination therapy and therefore antibody-IL2 treatment would not lead to the recovery of T_{reg} activity.

### Example 2

### Optimization of Dosing and Further Characterization of the Immune Response Induced by Combined Treatment with an Anti-Cd25 Antibody and an Antibody-IL2 Fusion Protein

The dramatic effect seen in the experiment of Example 1 suggested that the therapeutic index of KS-ala-IL2 could be increased by combination therapy with an anti-CD25 antibody, allowing for a less frequent dosing of KS-ala-IL2. To test this, the effect of KS-ala-IL2 treatment on immune cell populations, with or without the anti-CD25 antibody PC61, was compared in 7-8 week old female C57BL/6 mice (n=3 per treatment group). Mice were treated intravenously with KS-ala-IL2, either with a single dose on day 3 or with two doses on day 3 and day 5, at a dose of 20 micrograms/mouse. In one experimental condition, groups of mice were treated in addition intraperitoneally with the PC61 antibody at a dose of 100 micrograms/mouse on day 0 and day 5, whereas in the other experimental condition, groups of mice did not receive PC61. Control groups received either only the PC61 antibody treatment on the schedule described above, or 0.2 ml/mouse PBS intraperitoneally at day 0 and day 5 and intravenously at day 3 and day 5.

Immune cell populations were analyzed by standard techniques, from blood samples collected on day 8, day 14, and day 21, using flow cytometry and antibodies to cell surface receptors CD4, CD8, CD25, and NK-1.1. A further blood sample was collected on day 10, and immune cell populations were analyzed by flow cytometry using antibodies against cell surface receptors CD8, CD44, CD62 and CD122, which identify CD8+ memory T-cells. The analysis was performed according to standard procedures familiar to those skilled in the art.

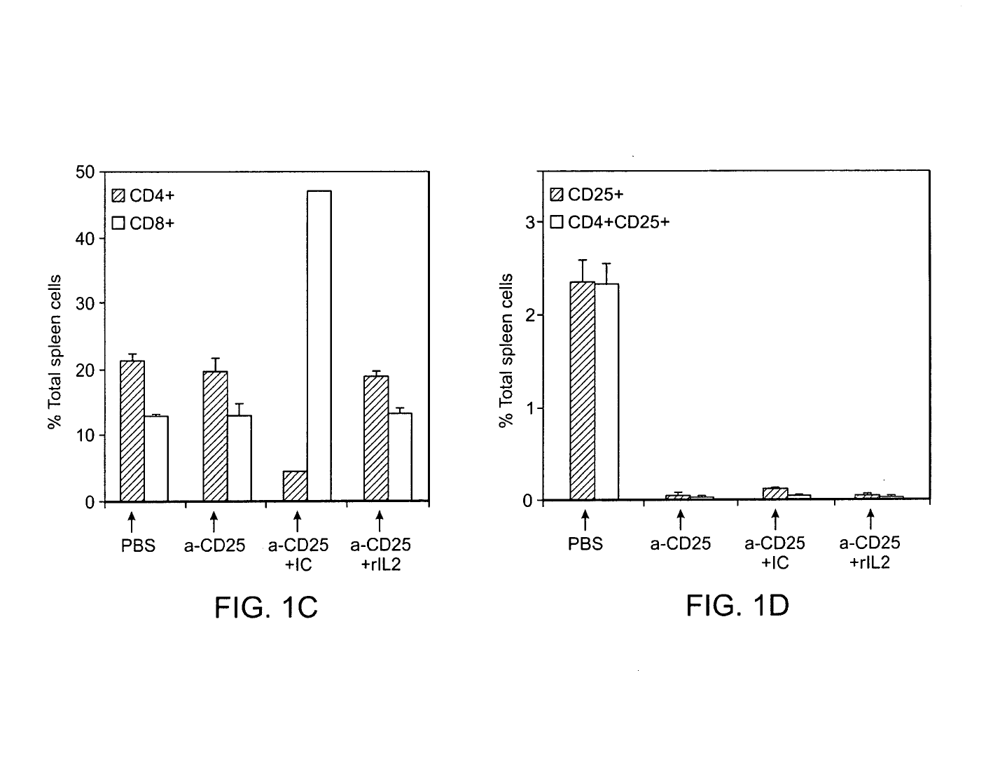

As expected, it was found that the PC61 antibody treatment caused about a five-fold reduction of the population of detectable CD25+ cells (data not shown), and about a 30-fold reduction of the population of detectable CD4+CD25+ cells (FIG. 2B), whereas the total populations of CD4+, CD8+ or NK-1.1+ (natural killer) cells were largely unaffected (FIG. 2C). The population of detectable CD4+CD25+ cells remained at about its reduced level throughout the duration of the experiment, as late as day 21 (FIG. 2B).

Treatment with the KS-ala-huIL2 alone caused a slight dose-dependent increase in the population of CD8+ cells (FIG. 2A) as well as of the CD4+CD25+ cells (FIG. 2B) at day 8, reaching approximately twice basal level, but by day 14 these populations had returned to basal level.

The population of NK1.1+ cells had increased approximately three-fold on day 8 (FIG. 2C). In contrast the effect of the combined treatment were profound: at day 8, the detectable CD4+CD25+ cell population was reduced approximately 50-fold relative to controls (FIG. 2B), and total CD8+ cell populations (FIGS. 2A and 2C) and NK1.1+ cell populations (FIG. 2C) had increased in a dose dependent manner, by seven-fold and 40-fold, respectively. In addition, the population of total CD4+ cells had decreased in a dose dependent manner by about 40% relative to controls (FIG. 2C).

On day 10, CD8+ cell population in the combination therapy groups was decreasing, compared to its level on day 8, but was still above the level of the treatment groups that only received KS-ala-IL2, and returned to base level by day 14 (FIG. 2A). The majority of these cells expressed cell markers found on memory T cells, i.e., those expressing high levels of CD44, CD62L and CD122 (the intermediate-affinity IL-2 receptor). Thus, most of the expanded CD8+ cells were of the memory phenotype (FIG. 2D). The number of naïve CD8+ T cells, on the other hand, did not vary between the treatment groups and remained low (FIG. 2D).

These results suggest that the combination is effective in transiently increasing the proliferation of CD8+ T cells and NK-1.1+ cells, while in addition markedly reducing the activity of a detectable T_{reg} (CD4+CD25+) population, at doses of KS-ala-IL2 that on their own do not produce such a pronounced effect on the CD8+ and NK1.1+ cell populations.

### Example 3

### Activity of Antibody-Cytokine Fusion Proteins Containing Monomeric or Dimeric IL-2, Combined with an Anti-CD25 Antibody, on Immune Cells

To determine whether the dimeric nature of IL-2 in KS-ala-IL2 was important for the dramatic effect on T cell and NK cell proliferation, additional forms of antibody-IL2 fusion proteins were tested. One such molecule, KS-ala-monoIL2, contains only a single IL-2 moiety, attached to the C-terminus of one of the two antibody heavy chains comprising the antibody moiety. An Fc-IL2 fusion protein dimeric for IL-2 and having an alanine between the Fc C-terminus and the IL-2 portion was also tested to determine the necessity for a whole antibody structure within the fusion protein. The alanine was inserted to increase the circulating half-life of the Fc fusion protein to the same degree as reported for huKS-ala-IL2 (Gillies et al., (2002) Clin. Cancer. Res., 8:210-216).

An experiment as described below was performed to compare the effectiveness of KS-ala-monoIL2 relative to KS-ala-IL2, in combination with anti-CD25 antibody PC61, in promoting the proliferation of immune cells.

To obtain KS-ala-monoIL2, a vector, was constructed containing separate expression cassettes encoding a KS-ala-IL2 heavy chain fusion protein, a KS antibody heavy chain, and the KS light chain. This expression vector was transfected into the myeloid cell line NS/0, and the fusion proteins were purified from conditioned cell culture media by binding to and elution from protein A Sepharose. The heterodimeric KS-ala-monoIL2 was further purified by SEC chromatography, and its identity was confirmed by non-denaturing and denaturing gel electrophoresis under reducing conditions. With respect to pharmacokinetics, it was observed in mice that circulating half-life of KS-ala-monoIL2 was at least as long as that of KS-ala-IL2.

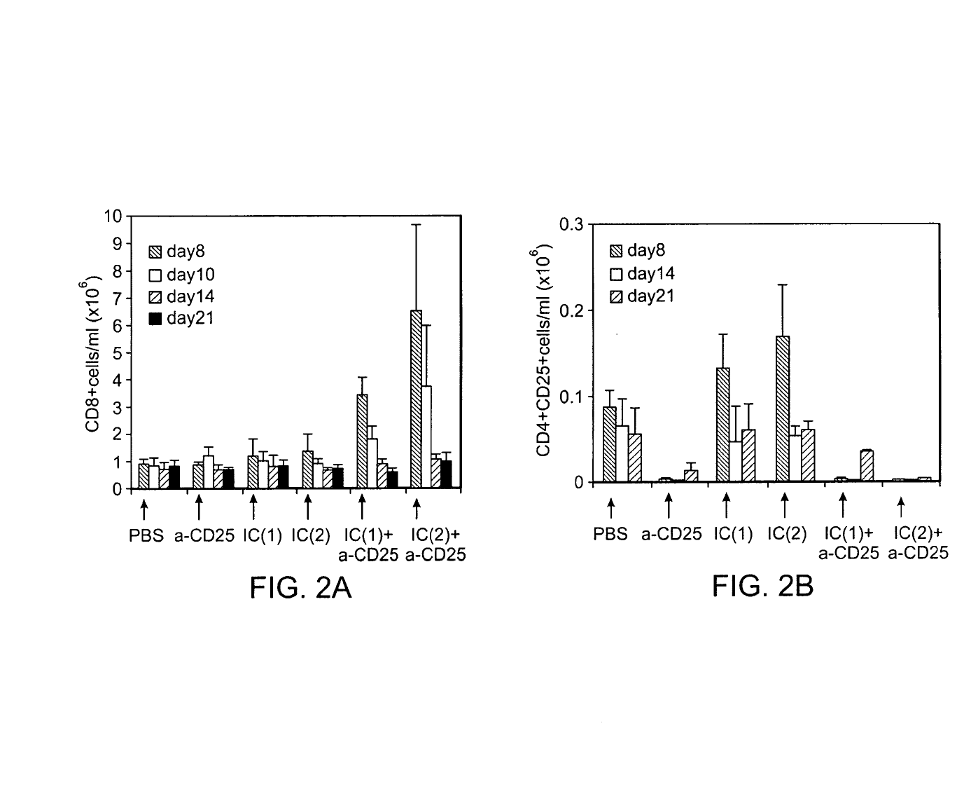

Two sets of C57BL/6 mice, divided into five groups each (n=3 per group) were treated with either 100 μg of PC61 or with 100 μg of a non-specific rat antibody on day 0 and 5. Each group in both sets was injected additionally on day 3 and 5 with either PBS, 20 μg of KS-ala-IL2, 20 μg of KS-ala-monoIL2, Fc-ala-IL2, or free IL-2. On day 8, peripheral blood cells and splenocytes were analyzed by flow cytometry according to standard techniques. The cell counts from the flow cytometry results for both the control and experimental groups are shown in FIGS. 3A-F.

Referring to FIGS. 3A-F, combined treatment with PC61 and KS-ala-IL2 induced marked CD8+ and NK1.1+ cell expansions, and a reduction in total CD4+ cells in both the peripheral blood and spleen samples, while free IL-2 showed little or no change compared to the PBS control. This result is similar to that already observed from previously discussed experiments. In contrast, treatment with KS-ala-monoIL2, containing one IL-2 molecule but exhibiting a long circulating half-life compared to free IL-2, showed only a slight increase in CD8 cells when combined with the PC61 antibody. The effect of KS-ala-monoIL2 on NK cell numbers was far less than what was seen with the dimeric form. As for CD4 cells, KS-ala-monoIL2 had no effect in reducing CD4 cells in the peripheral blood sample, while levels of CD4 cells in the spleen sample were only slightly less than the PBS control. Results with Fc-ala-IL2 showed a similar pattern of expansion for NK and CD8 cells as was seen for KS-ala-IL2. Overall, the combined percentage of CD8 and NK cells in the spleen increased from less than 20% in control animals to more than 75% in the KS-ala-IL2 and PC61 antibody combination group.

These results demonstrate that, in the context of this combined treatment, the dimeric nature of IL-2 in KS-ala-IL2 is important for its immunostimulatory properties and differentiates it from normal recombinant human IL-2 (rhIL-2 or “free IL-2”). Without wishing to be bound by theory, it is possible that the modest effect seen on NK cell proliferation with KS-ala-monoIL2 is mediated through the Fc moiety of KS-ala-monoIL2, since NK cells are known to express FcγR proteins.

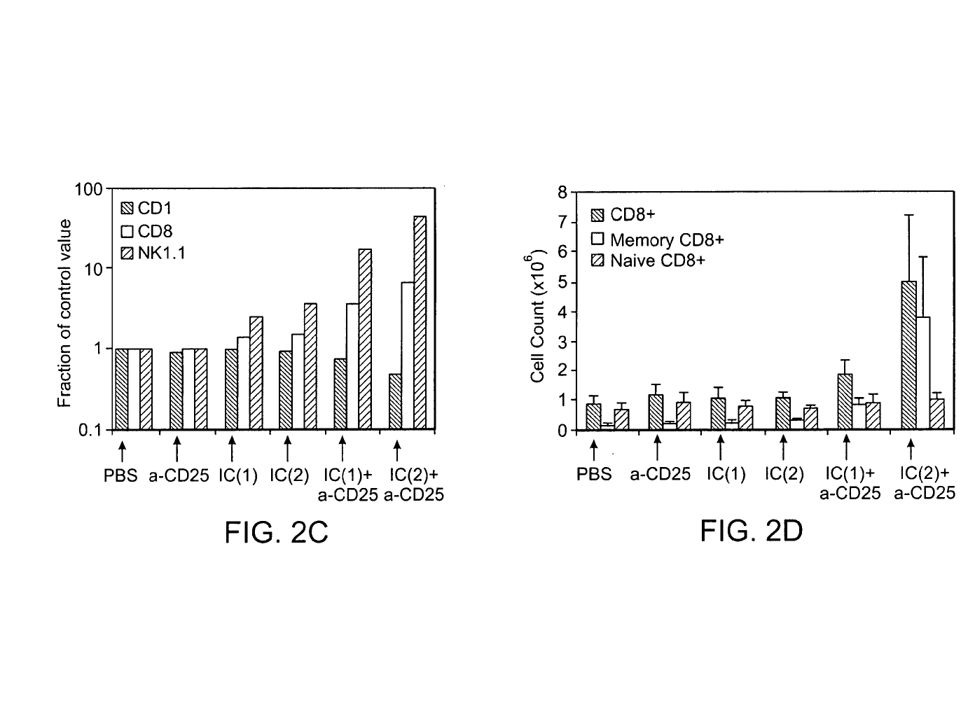

Splenic CD4 cells resulting from combination treatment with anti-CD25 antibody and KS-ala-IL2, KS-ala-monoIL2, IL-2, or Fc-ala-IL2 in the above experiment were further analyzed for expression of FoxP3 and CD25. Anti-FoxP3 and anti-CD25 antibodies were used. In this case, the 7D4 rat anti-mouse CD25 antibody, which binds to a discrete epitope from that of PC61, and has been shown by others to detect this receptor in its presence (Sauve et al., (1991), Proc. Natl. Acad. Sci. USA, 88:4636-40) was used. Total CD25+FoxP3+ cells were measured as a percentage of total splenocytes in mice treated with the indicated proteins. The reported percentage of double-positive cells below is based on the number of CD4 cells, which was much lower for the combination group. The data are shown in FIG. 4.

Treatment with anti-CD25 antibody clearly reduced the level of expression of CD25 on FoxP3+ spleen cells in the PBS and rIL-2 groups (FIG. 4A). In contrast, the groups treated with any of the dimeric IL-2 fusion proteins had increases in the percentage of FoxP3+ cells in the absence of anti-CD25 antibody, as well as continued expression of CD25 on FoxP3+ cells in the presence of PC61 antibody. Since the total number of CD4 cells in the spleen of mice treated with the combination of huKS-ala-IL2 and PC61 antibody declined, relative to the huKS-ala-IL2 alone group, the percentage of CD4 cells that were double positive for FoxP3 and CD25 increased as a percentage of CD4 cells (FIG. 4B). However, the total number of FoxP3+CD25+ cells was actually less (FIG. 4A). It appears that binding of PC61 to surface CD25 functionally prevented T_{reg} control of CD8 and NK cell expansion following stimulation with dimeric IL-2 fusion proteins, despite expression of hallmark cell surface markers, CD4, CD25, and FoxP3. In support of this contention, a recent study has shown that T_{regs} are not depleted after PC61 administration but that purified CD4+FoxP3+GITR+ T cells from treated mice lack regulatory function (Fecci et al., (2005), Clin. Cancer Res., 12:4294-4305).

### Example 4

### Stimulatory Activity of Anti-CD25 Antibody and Antibody-IL2 Fusion Proteins Containing Mouse IL-2 or an IL-2 Variant with Reduced Binding to the Intermediate-Affinity IL-2 Receptor Complex

Because in vitro experiments had shown that PC61, used in these studies, inhibits the proliferation of mouse CTLL-2 cells induced by mouse IL-2 but not human IL-2 or KS-ala-IL2 (which contains the human IL-2 sequence), it is possible that the effect observed in mice is simply a consequence of the use of a human IL-2 protein in a xenogeneic setting, which is able to circumvent the action of PC61.

To test whether neutralization by PC61 of IL-2 action at the IL-2 receptor complex is important, mice were treated with either KS-ala-IL2 containing human IL-2 (KS-ala-IL2) or mouse IL-2 (KS-ala-mIL2). The experiment was performed essentially as described in the previous Examples. C57BL/6 mice (n=3 per treatment group) were injected with 100 micrograms/mouse of PC61 antibody or with 100 μg of a non-specific rat antibody on days 0 and 5, and with 20 micrograms of KS-ala-IL2, KS-ala-mIL2, or with PBS on days 3 and 5. Mice in control groups received 100 micrograms/mouse of an irrelevant rat anti-mouse antibody instead of PC61. On day 8, peripheral blood samples were taken and analyzed by flow cytometry as before, using markers for CD4, CD8, NK1.1 and CD25.

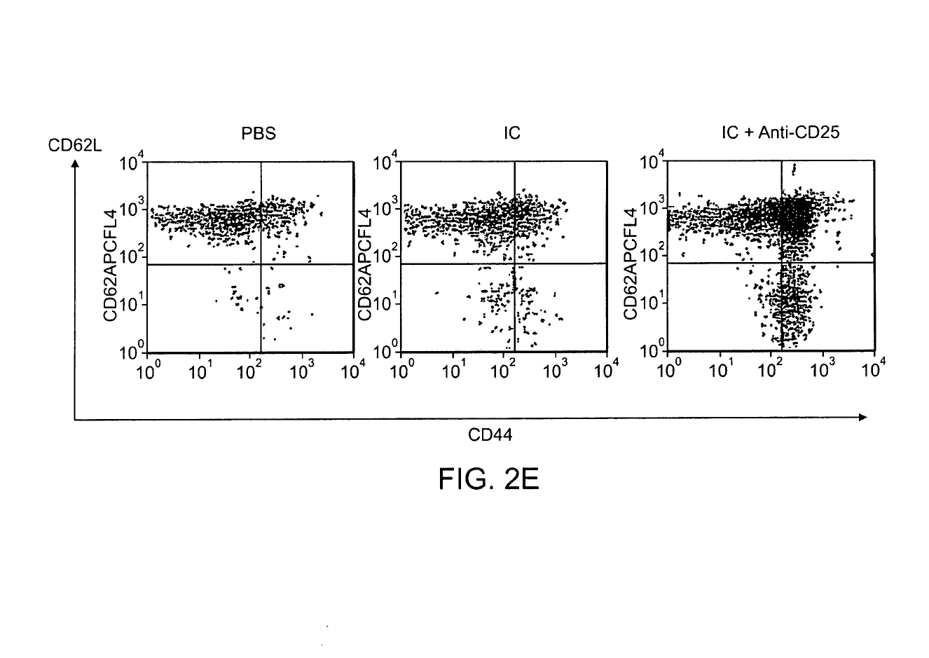

It was found that in the combination treatment the murine form of the antibody-IL2 fusion protein was just as active at inducing expansion of CD8+ and NK1.1+ cells in mice (FIGS. 5B and 5C), indicating that the effect was not related to the ability of the anti-CD25 antibody to neutralize cytokine bioactivity in vitro. However, the murine form of the antibody-IL2 fusion protein did not cause a reduction in the level of CD4+ cells (FIG. 5A).

In a second aspect of the experiment, the importance of signaling through the intermediate-affinity IL-2 receptor complex was assessed. For this purpose, an antibody fusion protein with an IL-2 variant that contains a single point mutation in IL-2 (D20T) necessary for binding the 13 chain of the IL-2 receptor is used. Previous studies have shown this to be highly selective (>1000-fold) for the high-affinity IL-2 receptor over the intermediate receptor (see, for example, U.S. Patent Application Publication No. 2003/0166163). A further group of C57BL/6 mice was treated with KS-ala-IL2(D20T), as above, on days 3 and 5. It was found that the CD8+ or NK1.1+ cell populations were not expanded when this variant was used (FIGS. 5B and 5C), indicating that signaling through the intermediate-affinity IL-2 receptor complex was required.

In summary, Examples 3 and 4 indicate that the invention minimally requires the use of a dimeric form IL-2 capable of signaling through the intermediate-affinity IL-2 receptor complex and an anti-CD25 antibody; however, it does not appear to require that the antibody be able to neutralize the binding and signaling of the exogenously added IL-2 to the high-affinity receptor complex.

### Example 5

### Reduction of T_{reg} Cell Activity and Enhancement of CD8+ T- and NK1.1+ Cells by Treatment with Non-Targeted IL-2 Fusion Proteins and an Anti-CD25 Antibody

In the preceding examples, antibody-IL2 fusion proteins were used; however, the antibody variable region, specific for EpCAM, was incidental to the observed effects on immune cell population changes and it is therefore likely that non-targeted forms of dimeric IL-2 fusion proteins, in combination with PC61, are equally effective as the preceding antibody-IL2 fusion proteins in their ability to enhance CD8+ cells and NK1.1+ cells, while reducing the activity of CD4+CD25+ cells.

The following experiment may be used to test this prediction. Examples of non-targeted dimeric IL-2 variants include an Fc-IL2 fusion protein, consisting of the Fc portion of human IgG1 fused to the N-terminus of human IL-2, or IL2-Fc, consisting of human IL-2 fused to the N-terminus of the Fc portion of human IgG1. Because it has been shown that IL2-Fc proteins maintain CDC and ADCC effector functions (see e.g., U.S. Pat. No. 5,349,053), while Fc-IL2 proteins do not, IL2-Fc is treated further with N-glycanase to remove the N-linked glycosylation at Asn297 to remove effector functions and avoid killing IL-2 receptor-bearing T cells. As comparators, KS-ala-IL2 and enzymatically deglycosylated KS-ala-IL2 (at Asn297) are used.

The experiment is performed essentially as described in the previous Examples. C57BL/6 mice (n=3 per treatment group) are injected with 100 micrograms/mouse of PC61 antibody on days 0 and 5, and with 20 micrograms of Fc-IL2, deglycosylated IL2-Fc, KS-ala-IL2, or deglycosylated KS-ala-IL2, or with PBS on days 3 and 5. Mice in control groups receive 100 micrograms/mouse of an irrelevant rat anti-mouse antibody instead of PC61. On day 8, peripheral blood samples are taken and analyzed by flow cytometry as before, using markers for CD4, CD8, NK1.1 and CD25.

According to the invention, it is seen that both non-targeting, dimeric IL-2 fusion proteins are approximately as effective as the KS-ala-IL2 fusion protein in reducing CD4+CD25+ cells and expanding both CD8+ T cells and NK1.1+ cells. Furthermore, abrogation of Fc receptor binding through deglycosylation of either IL2-Fc or KS-ala-IL2 has little effect on this process. Therefore, either Fc-IL2 or IL2-Fc (with Fc receptor binding removed through mutation, enzymatic treatment to remove the N-linked glycan, or through the use of isotypes with low Fc receptor binding, such as, for example, IgG2) are considered useful embodiments of the invention for systemic functional inactivation of T_{reg} cells and systemic expansion of CD8+ T-cells and NK cells when combined with an anti-CD25 antibody. (See U.S. Patent Application Publication No. 2003-01045294 for a discussion of removing Fc receptor binding in fusion proteins).

Interestingly, it had been observed that particular anti-IL2-antibody/IL-2 complexes, in which the antibody occludes the region of IL-2 involved in IL-2 receptor a interaction, are able to stimulate proliferation of memory CD8+ T-cell and NK cells in vivo (see Boyman et al., (2006), Science, 311:1924-1927). This effect was markedly reduced with the use of the corresponding F(ab)₂ fragment instead of the intact antibody, suggesting that for this protein composition, Fc/Fc receptor interactions are critical for the presentation of IL-2 to responder cells.

### Example 6

### Enhanced Anti-Tumor Activity of an Antibody—IL2 Fusion Protein when Combined with an Anti-CD25 Antibody

An experiment such as the one described in this example may be used to show that the effect of the combination treatment on immune cells correlates with a more efficacious anti-tumor treatment. For example, C57BL/6 mice are implanted subcutaneously with LLC/KSA tumor cells, a Lewis lung carcinoma cell line which is transfected to express the human cell surface protein EpCAM and is recognized by the KS antibody. The mice are then treated with KS-ala-IL2 in combination with the PC61 antibody. A non-targeted dimeric IL-2 fusion protein, such as Fc-IL2 serves as a control to assess the relative importance of targeting IL-2 to the tumor.

In the experiment described below, a treatment schedule as described in the previous Examples is used, but optionally other dosing regimens of the antibody and the IL-2 fusion protein may be used. 7-8 week old female C57BL/6 mice (n=6 per group) are implanted subcutaneously with LLC/KSA. When skin tumors reach an average of size of 50 mm³, the mice are treated, for example essentially as described in the previous Examples: on day 0 and 5, groups of mice are injected either with 100 micrograms/mouse of PC61 or with 100 micrograms/mouse of a non-specific rat antibody; on days 3 and 5, the groups of mice are further treated with 20 micrograms/mouse of KS-ala-IL2, or with 20 micrograms/mouse of Fc-IL2, or with PBS. On day 8, peripheral blood samples are collected and analyzed by flow cytometry as before, using markers for CD4, CD8, NK1.1, and CD25. Serial measurements of tumor volumes are also taken twice a week throughout the course of the experiment.

According to the invention, it is expected that the results will show that the CD25 antibody alone has little effect on the growth of this tumor and that two doses of KS-ala-IL2 alone have only some activity. Moreover, the combination therapy is expected to have a significant effect on tumor growth rate, compared to either agent alone, and this is expected to correlate with expansion of CD8+ T cells and/or NK1.1+ cells. In addition, it is expected that tumor targeting of IL-2 also plays an important role in anti-tumor activity since the treatment of animals with anti-CD25 and Fc-IL2 is expected to show less anti-tumor activity than the treatment with anti-CD25 and the targeted KS-ala-IL2 molecule.

Other dosing schedules may be selected and may be tested in a mouse model. For example, it may be useful to expand activated T-cells before administering the anti-CD25 antibody treatment, and an optional initial treatment with KS-ala-IL2 two or three days before administration of the standard dosing schedule described above can be included. The experimental dosing schedule can be further modified to interpose additional treatments to promote the activation of immune cells, or to generate effector cells, as desired. Optionally, other T-cell activating agents, such as CpG, may be included in the schedule.

In a further aspect of the experiment, the effector cell type largely responsible for the ant-tumor activity can be assessed by depletion of either CD8+ T cells or NK cells. On day 1 and 6, the two groups of mice receiving the combination therapy are further treated intraperitoneally with 100 micrograms/mouse of an anti-CD8 antibody or with 20 microliters/mouse of anti-asialo GM1 (#986-10001, Wako Chemicals USA, Richmond, Va.), and the mice are followed as described in this Example. If, for example, it is found that the treatment with anti-CD8 antibody results in mice with a significantly larger tumor burden, it would confirm that the CD8+ cells are an important effector cell population.

### Example 7

### The Effect of Anti-CD25 Antibody/Antibody-IL2 Combination Treatment on Anti-Tumor Activity in Another Experimental Metastasis Model

Since antibody targeting might exert its primary anti-tumor activity in the tumor microenvironment rather than as a consequence of effectors in the peripheral blood, the effects of combining a tumor-targeting immunocytokine with an anti-CD25 blockade were tested. The B16 melanoma model was chosen in order to facilitate comparing the results with those previously reported using anti-IL2 antibody and IL2 systemic gene therapy (Kamimura et al., (2006), J. Immunol., 177:1924-1927).

B16/KSA, a stably transfected B16 melanoma clone expressing the antigen for the huKS antibody (KSA or EpCAM, epithelial cell adhesion molecule) was generated by trans-infection using a retroviral vector as described in Gillies et al., (1998), J. Immunol., 160:6195-6203. The cells were cultured in a cell growth medium containing G418 (1 mg/ml) (Invitrogen, Carlsbad, Calif.). Mice were injected with 2×10⁵ viable single cells of B16/KSA in 0.2 ml PBS intravenously on day 0 and were allowed to recover for one day.

On days 1 and 5, the mice were injected intraperitoneally with either rat IgG or the anti-CD25 antibody PC61 at 100 micrograms/dose. huKS-ala-IL2 was injected intravenously on days 3 and 5 at 20 micrograms/dose. The mice were monitored for symptoms and were sacrificed when the control group became moribund, which occurred at day 21 after tumor implantation. Lungs were removed, weighed, and fixed in Bouin's solution. Anti-tumor efficacy was evaluated by (a) lung weight normalized to body weight, and (b) percentage of lung surface covered by metastasis.

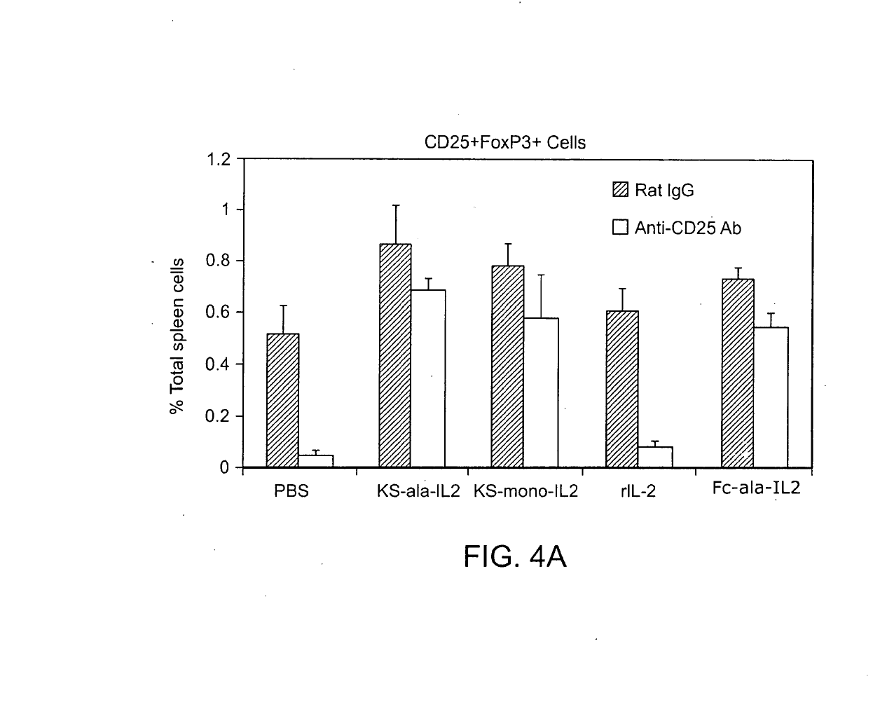

Tumor burden in mouse lungs was determined in two ways. The percentage of lung surface covered with tumor was estimated by visual inspection and represent the average of the group of 6 animals+/−the standard error. Tumor burden was also determined by weighing the lungs and normalizing the values to the body weight of the individual mouse. The difference between the combination group and the BPS control group was statistically significant by both determinations (p<0.01) but the difference with the huKS-ala-IL2 group was not significant. Data for % surface metastases and tumor burden are depicted in FIG. 7.

As shown in FIG. 7, treatment with huKS-ala-IL2 with the control rat IgG had a measurable effect on tumor burden, but the difference was not statistically significant without the addition of the anti-CD25 antibody. The difference between the huKS-ala-IL2 monotherapy and combination therapy groups was not quite statistically significant due, in part, to the variability in response of the individual mice.

A possible explanation of these result is that anti-CD25 blocks not only T_{reg} function on CD4+CD25+ cells, but also effector function on CD8+CD25+ cells. In an attempt to circumvent this, the alternative approach in Example 13 was tried. Further, the differences between these data and those reported earlier by Kamimura et al. ((2006), J. Immunol., 177:1924-1927) could be explained by the timing of treatment as well as the time of measurement of tumor burden (day 12 vs. day 21). One might expect differences in non-curative treatments to be quickly lost as tumor burden in all groups of animals increases. In fact, conditions of immune stimulation with huKS-ala-IL2 alone were already quite potent at preventing outgrowth of established B16 lung metastases and may easily be made more effective by dosing more than two days. This high potency may be a reflection of the targeting effect of antibody-IL2 fusion proteins, which has been shown in all models tested to date to be far more potent than free antibody and cytokine (Davis et al., (2003), Cancer Immunol. Immunother., 52:297-308.

### Example 8

### The Effect of Combining a Dimeric IL-2 Fusion Protein and an Anti-CD25 Antibody Together with a Vaccine to Reduce T Regulatory Cells and Enhance the Expansion of CD8 Positive T Cells

To demonstrate the value of treatment with anti-CD25 antibody and a dimeric IL-2 fusion protein in the context of a vaccination, the following experiments are performed. Mice are first pre-treated with an anti-CD25 antibody or a control vehicle solution, for example on days 0 and 5 of the experiment. An antigen, optionally including an adjuvant, is then administered, for example on day 1. A useful antigen for monitoring CD8+ T cell responses is the AH1-Ala5 peptide recognized by class I MHC and presented by syngeneic tumors in Balb/c mice (Slansky et al., (2000), Immunity, 13:526-538). This can easily be administered with incomplete Freund's adjuvant. In the present example, dimeric IL-2 fusion protein is administered on days 3 and 5 (20 μg/mouse) by intravenous injection. On day 11 and on day 18, groups of mice are sacrificed and their spleen cells are analyzed for immune cell subsets, as well as by ELISPOT for enumeration of the number of AH1-Ala5 peptide specific CD8+ T cells expressing interferon gamma (IFN-γ). This ELISPOT assay is well known in the art for measuring antigen-specific cytotoxic CD8 cell levels (CTLs) (see, e.g., Power et al., (1999), J. Immunol. Meth., 227:99-107). Results show that the combination of anti-CD25 antibody and dimeric IL-2 fusion protein added to a vaccination protocol increases the number of antigen-specific CD8+ T cells to a greater extent than any of the individual agents alone.

While this specific protocol is shown to enhance a CD8+ cell vaccine response to immunization, many variations of this approach can be envisioned as embodiments of the invention. For example, in one embodiment, the dimeric IL-2 molecule (e.g. Fc-IL2 or IL2-Fc) is administered at the same time as the antigen or within about 24 to 48 hours, and preferably at a distant site from where the emulsified adjuvant is injected. While the anti-CD25 antibody is preferably injected intravenously, the dimeric IL-2 protein can be administered by several alternative ways. Intravenous injection can be used, as described in the examples given above, but subcutaneous or intra-muscular injection can be used as well. Another delivery method can include injection of a DNA vector encoding a dimeric IL-2 fusion protein.

Alternatively, a protein such as a CEA-Fc-IL2 (SEQ ID NO:9) fusion protein is administered, with CEA being considered the antigen and the Fc-IL-2 moiety having an adjuvant effect as well as providing dimeric IL-2. Administration of an anti-CD25 antibody is performed preferably before injection of the fusion protein but can range from 0 to 2 days before. Optionally, this procedure is repeated to provide a boosting effect. A cellular immune response is then monitored by standard techniques.

### Example 9

### The Effect of CD4 Depletion on NK and CD8 T Cell Proliferation Resulting from Treatment with Antibody-IL-2 and an Anti-CD4 or Anti-Cd25 Antibody

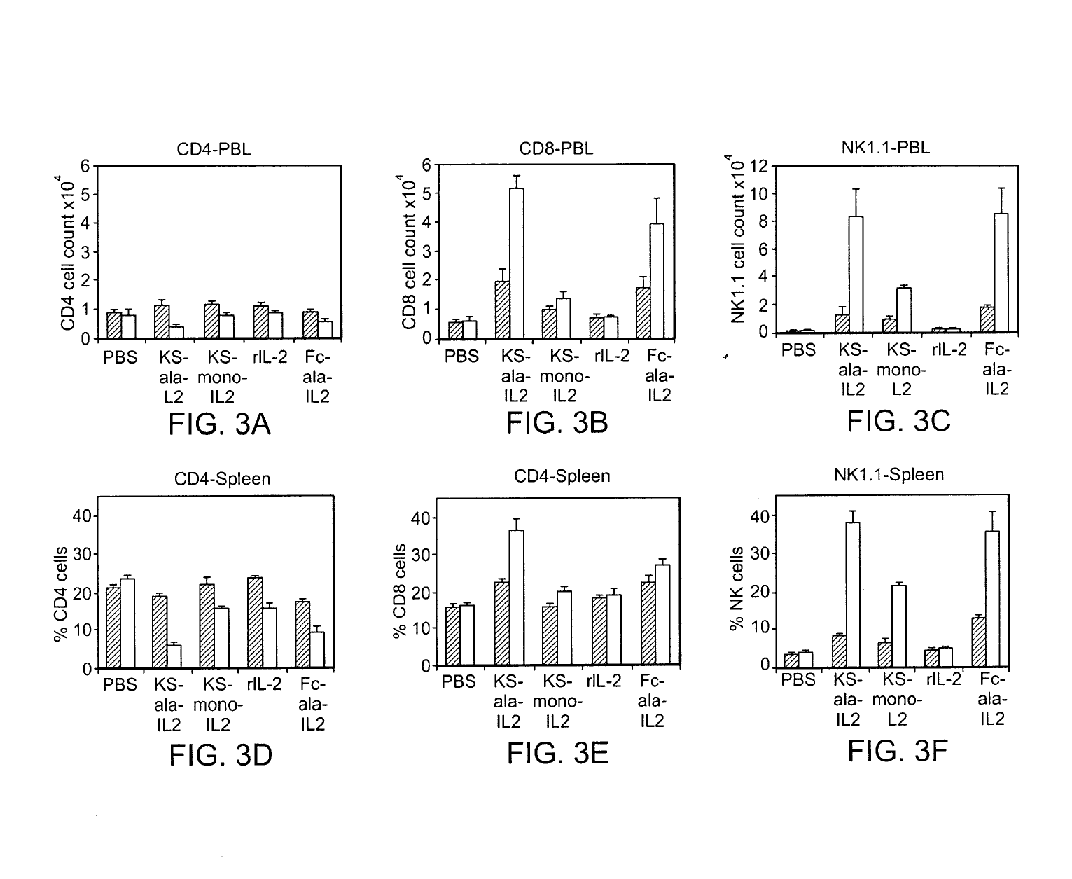

The results of the previously described experiments indicate that functional blockade of T_{reg} cells by anti-CD25 antibody allows for a more potent stimulation of both NK and CD8 T cell proliferation by dimeric IL-2 antibody fusion proteins, suggesting that these cells actively suppress this process in vivo. Since anti-CD4 antibody depletion might be expected to remove this inhibition as well, the effects of these two antibody approaches were compared in combination treatment with huKS-ala-IL2. According to the same dosages and scheduling as in the previously described experiments, mice were treated with either anti-CD4 antibody or anti-CD25 antibody on days 1 and 5, and huKS-ala-IL2 on days 3 and 5. Whole blood was then obtained by retro-orbital bleeding and analyzed by flow cytometry. Cell counts derived therefrom are depicted in FIGS. 6A-E.

As shown in FIGS. 6A-E, treatment with anti-CD4 antibody under the conditions used resulted in near-complete elimination of CD4 and CD4+CD25+ cells on the day of analysis, day 8 (FIGS. 6A and 6B). Combination therapy with either antibody resulted in similar increases of CD8 cell expansion, suggesting that functional blockade of T_{regs} or elimination of all CD4 T cells had a similar effect. One significant, but not unexpected, difference was that CD8 cells treated with huKS-ala-IL2 and anti-CD4 had much higher levels of CD25 than the combination group treated with anti-CD25.

Another interesting difference between the combination treatment groups was that depletion of CD4 cells did not result in the same level of expansion of NK cells by huKS-ala-IL2 that was observed with the anti-CD25 antibody, although CD4+CD25+ cells were depleted by this treatment (FIG. 6A-E). Instead, the increase in the NK1.1+ population was similar to what was seen with huKS-ala-IL2 alone (FIG. 6E).

Other potential immune cell interactions were tested by administering additional depleting antibodies (anti-CD8 and anti-GM1 to deplete NK cells) to mice dosed with huKS-ala-IL2 and PC61. The addition of these antibodies was completely effective at removing the respective cell types in mice that otherwise would have greatly expanded numbers in response to huKS-ala-IL2 and PC61 (FIG. 6A-E). The effect of adding the anti-CD8 antibody to mice dosed with huKS-ala-IL2 and PC61 was a slight but not statistically significant reduction in NK cells. In contrast, depletion of NK cells with anti-GM1 completely reversed the stimulatory effect of adding PC61 to mice dosed with huKS-ala-IL2 with respect to CD8 cell expansion.

The use of anti-CD4 as a means of reducing T_{reg} activity also resulted in a increase of CD8+CD25+ T cells. These CD8+CD25+ T cells may have more potent effector activity since the means of reducing inhibition did not interfere with CD25 activation of these cells. In this case, only CD8 cell proliferation was enhanced, whereas CD4, NK and Gr1+ cells were all enhanced using the mutated antibody-IL2 construct, as described in Example 13 below.

That CD4 depletion stimulated the expansion of CD8 cells but not NK cells and to a slightly less extent than anti-CD25 antibody, may be due to the fact that NK cells appear to be required for optimal expansion of CD8 cells in mice co-administered with the IL-2 antibody fusion protein and anti-CD25 antibody.

### Example 10

### Effects of Anti-Cd25 Antibody and huKS-IL2 in SCID and Cd4-Depleted B1/6 Mice

In order to test whether CD4 cells are required for the expansion of NK cells induced by huKS-ala-IL2 and PC61 antibody administration, experiments were undertaken in CD4-depleted B1/6 mice and SCID CB17 mice lacking functional T and B cells.

On days 1 and 5, SCID mice were injected intraperitoneally with either the control antibody rat IgG or the anti-CD25 antibody PC61 (100 micrograms/dose, diluted in PBS to a total volume of 200 microlitres). On days 3 and 5, those mice having received rat IgG were then dosed intravenously through the tail vein with either PBS or huKS-ala-IL2 (20 micrograms/dose, diluted with PBS to a total volume of 100 microlitres). Likewise, SCID mice having received the anti-CD25 antibody were dosed intravenously through the tail vein with either PBS or huKS-ala-IL2 (20 micrograms/dose).

On days 1 and 5, B1/6 mice were injected intraperitoneally with either the control antibody rat IgG, the anti-CD25 antibody PC61, the anti-CD4 antibody GK1.5, or both PC61 and GK1.5 (100 micrograms/dose, diluted in PBS to a total volume of 200 microlitres). On days 3 and 5, the mice were then dosed with either PBS or huKS-ala-IL2 (20 micrograms/dose, diluted with PBS to a total volume of 100 microlitres).

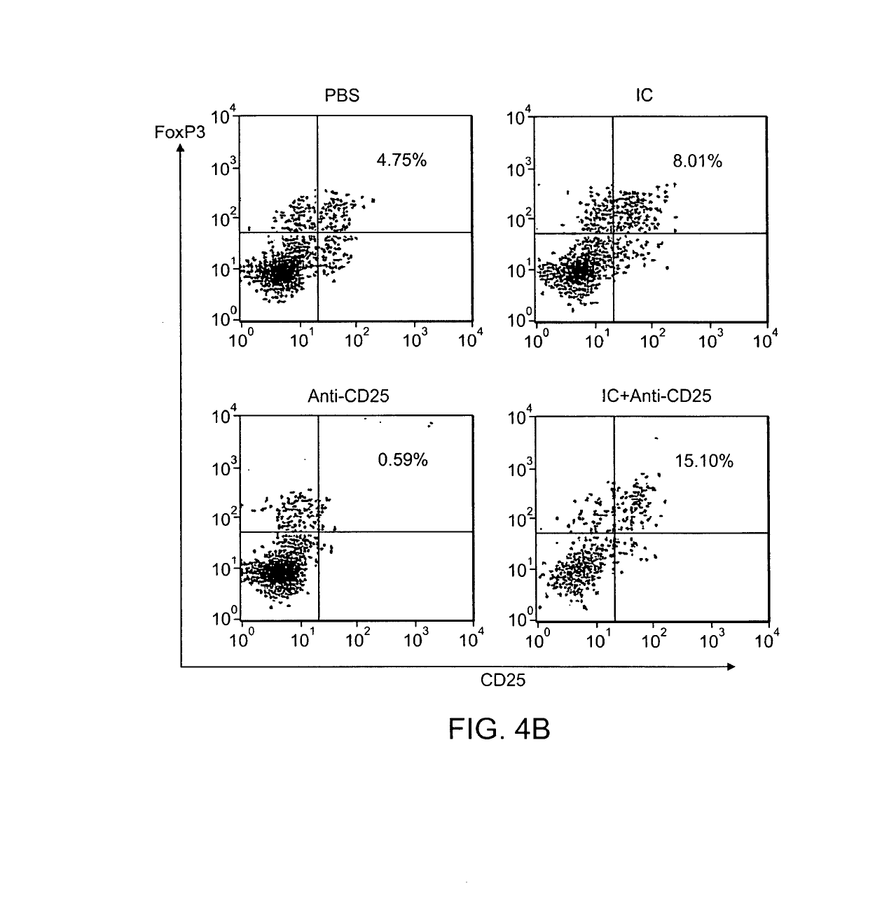

Peripheral blood samples were taken and whole blood cells were analyzed by flow cytometry on day 8. Blood cells from SCID mice were evaluated for levels of DX5+ NK cells, CD11 b and Gr1 (granulocytes). Blood cells from B/6 mice were evaluated for NK1.1+ NK cells (FIG. 8C) and CD8+ T cells (FIG. 8D).

Surprisingly, the addition of anti-CD25 antibody resulted in a dramatic expansion of DX5+ (NK) cells, relative to the huKS-ala-IL2 alone treatment group, and the majority of these were CD11b+ indicating a mature phenotype (FIG. 8A). Gr1+ cells were also expanded more than 10 fold as well in the combination group (FIG. 8B). These results show that depletion of CD4 cells was not the reason that NK cells did not expand in response to huKS-ala-IL2 and anti-CD25 antibody in the previous experiment, but rather the lack of blockade of CD25, apparently on a different cell type. Overcoming this regulatory mechanism led to the expansion of multiple lymphocyte and granulocyte populations in response to dimeric IL2.

Consistent with that observed in SCID mice (lacking functional CD4 cells), the addition of PC61 to CD4-depleted, immune competent mice restored the high level of NK cell proliferation induced by huKS-ala-IL2 (FIG. 8C). Unlike NK cells, CD8 T cell numbers in the same mice increased as a consequence of either anti-CD4 or anti-CD25 antibody treatment, as observed in the earlier experiment (FIG. 8D). Together these data suggest that CD4 cells (presumably CD4+CD25+ T_{regs}) limit CD8 T cell expansion while NK cell expansion is regulated by another cell type also expressing and functionally dependent on CD25 for its regulatory capacity, but not of T cell lineage.

### Example 11

### Treatment of Human Cancer Patients with an Immunocytokine and an Anti-CD25 Antibody

According to the invention, human cancer patients are treated with an anti-CD25 antibody and with an IL-2-containing immunocytokine. Proper dosing order can be established in mouse tumor models in experiments as described in the Examples above, and confirmed by subsequent testing in monkeys using the same reagents intended for human use. An exemplary treatment is as follows. A patient deemed suitable for immunocytokine therapy is first treated with a human anti-CD25 antibody at the dose recommended by the manufacturer. Such antibodies are known in the art and are already marketed for use in prevention of graft rejection (for example daclizumab, also known as Zenapax® (Roche), or basiliximab, also known as Simulect® (Novartis)). For example, daclizumab is standardly administered at 1 mg/kg, intravenously. Administration is generally by infusion in a volume of 50 milliliters of a sterile 0.9% saline solution.

About 0 to about 72 hours after administration of the anti-CD25 antibody, an immunocytokine such as KS-IL2 (SEQ ID NOS: 2 and 3) or hu14.18-IL2 (for example, SEQ ID NO:7 and 8)(see, e.g., U.S. Patent Application Publication No. 2004/0203100 and Osenga et al., (2006), Clin. Cancer Res., 12(6):1750-1759) is administered by intravenous infusion. Typically a four-hour infusion is used, although a shorter or longer period of infusion may be used. An immunocytokine dose between 0.04 and 4 mg per square meter of body surface area is generally used, corresponding to about 0.1 to 10 mgs for an adult human patient. Optionally, a second dose of daclizumab is administered approximately 5 days following the first dose, together with a second dose of immunocytokine. Other dosing schedules may be used as appropriate, based on further pre-clinical and early clinical testing.

### Example 12

### Treatment of Human Cancer Patients with an Anti-Cancer Vaccine and the Combination of a Dimeric IL-2 Fusion Protein and an Anti-CD25 Antibody

According to another aspect of the invention, human cancer patients are treated with an anti-cancer vaccine, an anti-CD25 antibody and an IL-2 protein composition. Proper dosing order can be established in mouse tumor models in experiments as described in the Examples above, and confirmed by subsequent testing in monkeys using the same reagents intended for human use. An exemplary treatment is as follows. A patient deemed suitable for cancer vaccine therapy is treated with an anti-CD25 antibody such as daclizumab)(Zenapax® at the dose recommended by the manufacturer, such as 1 mg/kg intravenously. Administration is generally by infusion in a volume of 50 milliliters of a sterile 0.9% saline solution. About 0 to about 72 hours after administration of the anti-CD25 antibody, a cancer vaccine is administered. For example, a cancer vaccine composed of a cocktail of survivin-derived peptides is administered which elicits an immune response to tumors expressing the tumor-selective antigen survivin. (See U.S. Patent Application Publication No. 2004/0210035). The dose is about 100 micrograms per peptide, and the rout of administration is by subcutaneous injection. Optionally, a boost cycle is performed using the same treatment protocol. Shortly thereafter a dimeric IL-2 fusion protein is administered either by intravenous or subcutaneous injection of the protein or alternatively, by injection of a vector encoding such protein, for example Fc-IL2 or IL2-Fc fusion proteins. Optionally, the Fc portion of the fusion protein is modified so that it does not elicit antibody effector functions such as CDC or ADCC that could blunt the T cell response. For vaccination procedures, dosage and route of administration of the vaccine are generally unchanged from procedures that do not include anti-CD25 antibodies.

Alternatively, according to another embodiment, a human cancer patient is first treated with one round of a cancer vaccine, followed by the dimeric IL-2 fusion protein, to initiate the induction phase of an immune response, and thereafter, a second round of treatment is initiated with an anti-CD25 antibody, as described above. For example, in one embodiment, the patient is pretreated with cancer vaccine. One to seven days following the pretreatment, the patient is then treated with an IL-2 protein composition and an anti-CD25 antibody. In an alternate embodiment, the patient is pretreated with cancer vaccine. One to seven days following pretreatment, the patient is then treated with an IL-2 protein composition. One to seven days following the treatment with the IL-2 protein composition, the patient is then treated with an anti-CD25 antibody. Optionally, before treatment with the anti-CD25 antibody, the patient is given a boost treatment of the cancer vaccine.

### Example 13

### Comparison of the Effects of Fusion Proteins Containing Mutant Versions of IL-2 with Immunocytokines Containing Wild-Type IL-2 and Anti-CD25 Antibodies

Without wishing to be bound by theory, the hyperproliferation of immune cells induced by the combination of an antibody-IL2 fusion protein and anti-CD25 antibody appears to be due to the stimulation of the intermediate affinity IL-2 receptor while simultaneously blocking CD25. Therefore, as an alternative to the combination of anti-CD25 antibodies and IL-2-containing fusion proteins which block CD25 with an antibody, antibody-cytokine fusion proteins containing a mutant IL-2 with a defect in the IL-2Rα binding surface were tested for their effects on T cell levels to see if similar effects could be achieved.

The amino acid residues R38 and F42 of IL-2 both interact with the receptor a chain (Sauve et al., (1991), Proc. Natl. Acad. Sci. USA, 88:4636-40; Heaton et al., (1993), Cell Immunol., 147:167-179). Therefore, a version of huKS-ala-IL2 with mutations of both residues (R38W and F42K) was engineered to effectively block the interaction with CD25 (referred to as “huKS-ala-IL2RF”). As a control, position D20 of IL-2 was mutated to threonine (referred to herein as “D20T” or “D”) (huKS-ala-IL2D20T, also referred to as huKS-ala-IL2D) in order to block binding to CD122, while retaining binding to the αβγ high affinity receptor complex. In both cases, the mutant antibody-IL2 fusion proteins are capable of inducing proliferation through the other IL-2 receptor form (Hu et al., (2003), Blood, 101:4853-61).

A group of 7 week-old, female Balb/C mice were divided into two groups, an experimental group and a control group, with subgroups of three mice each. On days 1 and 5, the experimental mice were administered 100 micrograms of the anti-CD25 antibody PC61, while control mice were administered 100 micrograms of the control antibody rat IgG. On days 3 and 5, the subgroups of the experimental and control groups were each administered one of PBS, KS-ala-IL2, KS-ala-IL2(R38W, F42K) (also referred to as KS-ala-IL2RF), or KS-alaIL2(D20T) in the amount of 20 micrograms/mouse. On day 8, the animals were sacrificed and blood cells and splenocytes were analyzed for lineage markers and IL-2 receptor expression.

The table below shows the surface markers that were analyzed, and the number of cells per milliliter of blood that were counted.

TABLE 1

| | Treatment | | | | | | | |
|---|---|---|---|---|---|---|---|---|
| Surface | | | Rat IgG + | Rat IgG + | | Anti- | Anti-CD25 + | Anti-CD25 + |
| marker | Rat | Rat IgG + | KS-ala-IL2 | KS-ala-IL2 | Anti- | CD25 + | KS-ala-IL-2 | KS-ala-IL2 |
| (cells/ml) | IgG | KS-ala-IL2 | (R38W, F42K) | (D20T) | CD25 | KS-ala-IL2 | (R38W, F42K) | (D20T) |
| CD8+ | 0.5485 | 1.1097 | 7.9899 | 0.5277 | 0.6083 | 9.7626 | 10.5358 | 0.5831 |
| CD122+ | 0.3714 | 1.6089 | 20.0504 | 0.4348 | 0.3551 | 24.8544 | 22.9685 | 0.3948 |
| CD8+ | 0.1194 | 0.7089 | 7.9215 | 0.1207 | 0.1199 | 9.6991 | 10.2689 | 0.1299 |
| CD122+ | | | | | | | | |
| Gr1+ | 0.9634 | 2.0894 | 8.1877 | 1.0460 | 1.0259 | 9.4452 | 8.8174 | 1.0574 |
| Gr1^{hi}+ | 0.6821 | 1.3888 | 2.3932 | 0.7451 | 0.7848 | 2.3589 | 2.2953 | 0.8139 |
| NK-1.1 | 0.2068 | 0.7784 | 15.0629 | 0.2161 | 0.2059 | 16.7486 | 17.6347 | 0.2270 |

These results indicate that certain treatments of the invention caused a major effect on effector cell populations, such as cytotoxic T cells (represented by CD8), granulocytes (represented by Gr1), and natural killer cells (represented by NK-1.1). In particular, the numbers of these cells were significantly increased in mice treated with either KS-ala-IL2(R38W, F42K), anti-CD25 with KS-ala-IL2, or anti-CD25 with KS-IL2(R38W, F42K). Moreover, the impact of each of these three treatments on CD8+ cells, Gr1+ cells, and NK-1.1+ cells were not statistically different from each other, indicating that treatment with a single agent such as KS-ala-IL2(R38W, F42K) or with a combination such as KS-IL2 and an anti-CD25 antibody would achieve a similar useful immunostimulatory effect.

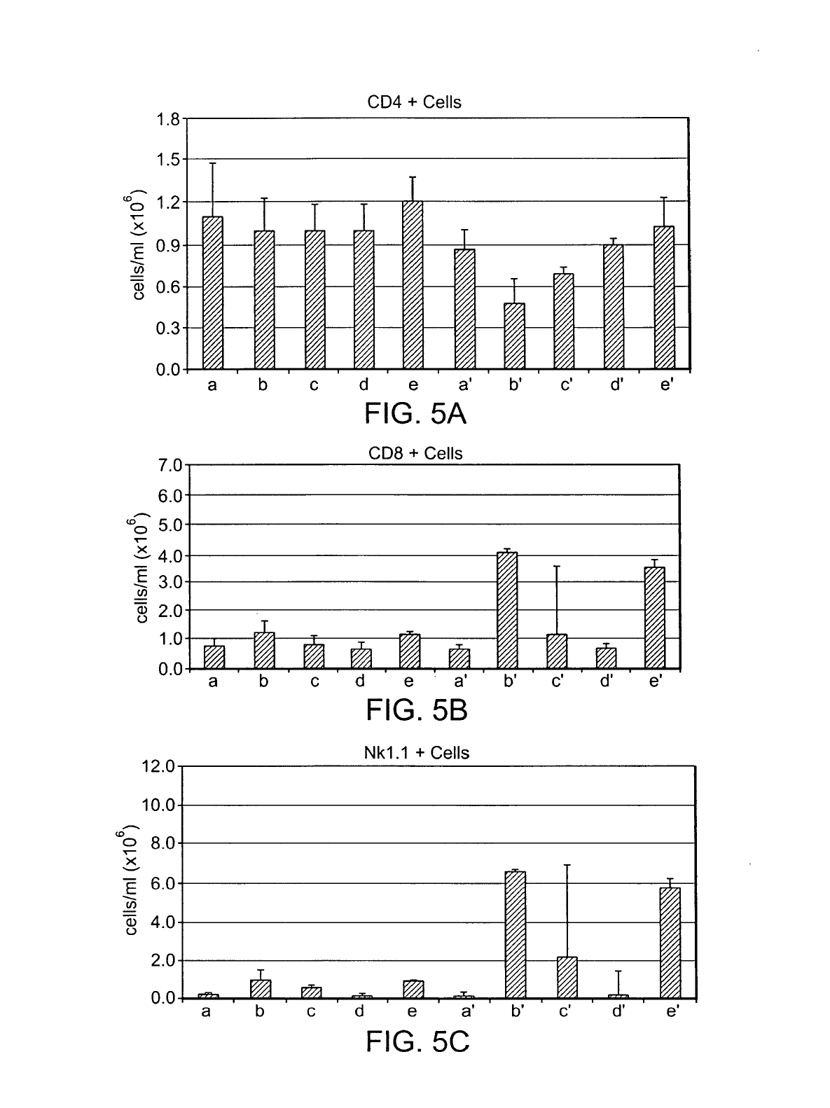

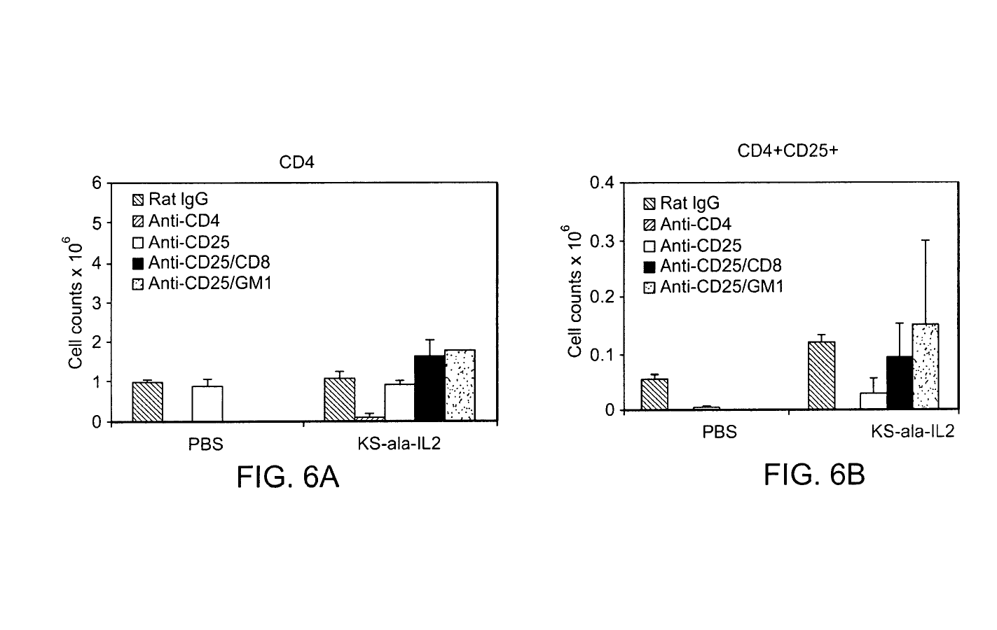

FIG. 9 also shows data for cell counts. Gr1+ cell counts include intermediate and high expressing subgroups, as well as NK1.1.+Gr1+ cells. As seen therein, immune cell numbers for all groups of animals receiving huKS-ala-IL2(D20T) (specific for the high affinity receptor only) were not significantly different from PBS control mice, even in the presence of the anti-CD25 antibody. This confirms that the proliferative responses to KS-ala-IL2, in combination with anti-CD25, are mediated through the CD122 receptor subunit. In striking contrast, huKS-ala-IL2(R38W, F42K) (specific for the CD122 receptor but not triggering CD25), induced a potent CD8 T cell (FIG. 10C), NK cell (FIG. 10E) and Gr1+ cell (FIG. 10F) proliferative response in the presence or absence of anti-CD25 antibody, whereas the wild-type molecule required anti-CD25 blockade for an enhanced response.

Unlike the KS-ala-IL2 and anti-CD25 combination therapy, huKS-IL2(R38W, F42K) monotherapy also stimulated the proliferation of CD4 T cells (FIG. 10A) and the majority of these cells expressed CD25 (FIG. 10B). This is likely explained by up-regulation of CD25 on these cells after stimulation via CD122 and subsequent response to endogenous IL2 that would otherwise be blocked by the anti-CD25 antibody or by T_{regs} (in the case of huKS-ala-IL2 alone). Evidence for this hypothesis is shown in the group receiving huKS-IL2(R38W, F42K) and the anti-CD25 antibody, in which case total CD4 T cell numbers decreased as a consequence of not being able to respond to endogenous IL2 capable of binding to the high affinity receptor.

Also of note, the group of mice receiving huKS-ala-IL2(R38W, F42K) as monotherapy was the only one to show a dramatic increase in CD8+CD25+ (presumably effector) cells (FIG. 10D). Again, this is likely due to the initial lack of suppression by T_{regs} (since huKS-ala-IL2(R38W, F42K) does not trigger CD25) under conditions that stimulate CD122 signaling by the antibody-IL2 fusion protein and, presumably, CD25 signaling by endogenous IL-2. On day 8, when the cells were analyzed immune cells by flow cytometry, the majority of the CD4+CD25 T cells also expressed FoxP3 (FIG. 9) and, therefore, likely had converted to a regulatory phenotype.

As these data show, unlike what was seen with the administration of antibody-IL2 fusion proteins with anti-CD25 antibodies, administration of huKS-ala-IL2(R38W, F42K) has the ability to strongly stimulate the intermediate IL-2 receptor without triggering CD25 and the consequent activation of T_{reg} function that otherwise limits proliferation. At the same time, CD25 is not blocked from functioning on CD4 and CD8 effector cells and both can be stimulated by endogenous IL-2. This may lead to more potent activation of CD8 effectors and proliferation (actually a modest reduction) of antibody-IL2 stimulated CD4 cells when huKS-ala-IL2(R38W, F42K) is co-administered with an anti-CD25 antibody that would block stimulation by endogenous IL-2. Apparently the endogenous IL-2 produced by the expanded CD4 population then triggers CD4+CD25 positive cells to up-regulate FoxP3 and convert to a suppressor phenotype.

Without wishing to be bound by theory, these results suggest that the compositions of the invention operate, at least in part, through an IL-2 moiety that interacts with the IL-2 receptor β subunit. This mechanism is implied by the observation, illustrated in Table 1, that the stimulation of effector cell populations seen for example, with KS-IL2 plus anti-CD25 is not observed with KS-IL2(D20T) plus anti-CD25. The D20T mutation is known to significantly reduce interaction between IL-2 and IL-2 receptor β.

Based on the observations in Table 1 above, the invention thus provides a number of therapeutic strategies for immunostimulation, based on the general principle that it is useful to inhibit the IL-2/IL-2Rα interaction and maintain the IL-2/IL-2Rβ interaction in targeted fusion proteins containing an IL-2 moiety. As illustrated in Table 1 above, this may be accomplished using an antibody against CD25, the IL-2Rα subunit, or by using a mutant form of IL-2 with reduced or abolished interaction with IL-2Rα. Alternatively, according to the invention, the same effect may be achieved by using an antibody or other protein that binds to the IL-2 fusion protein on the surface of IL-2 that interacts with IL-2Rα. Thus, the invention also provides compositions that include IL-2 fusion proteins combined with antibodies or other proteins that bind to IL-2 and block its interaction with IL-2Rα.

1. A method of enhancing the immunostimulatory effect of IL-2 in a patient comprising:
administering a CD25 antagonist and a protein comprising first and second IL-2 moieties, wherein the CD25 antagonist is an anti-CD25 antibody or portion thereof capable of binding to CD25, and
wherein the CD25 antagonist is administered in amount effective to enhance the immunostimulatory effect of the protein comprising an IL-2 moiety.

2. (canceled)

3. The method of claim 1, wherein the protein further comprises an immunoglobulin moiety.

4. The method of claim 3, wherein the immunoglobulin moiety comprises an antibody.

5. The method of claim 4, wherein the antibody comprises a variable region directed to an antigen presented on a tumor cell or in a tumor cell environment.

6. (canceled)

7. The method of claim 1, wherein the protein comprising first and second IL-2 moieties is capable of activating an intermediate-affinity IL-2 receptor complex.

8-10. (canceled)

11. The method of claim 1, wherein the anti-CD25 antibody is daclizumab or basiliximab.

12. The method of claim 1, wherein the CD25 antagonist is administered prior to administration of the protein comprising an IL-2 moiety.

13. (canceled)

14. The method of claim 1, wherein the effective amount of CD25 antagonist is between about 0.1 mg/kg and 10 mg/kg per dose.

15-16. (canceled)

17. The method of claim 1, wherein the effective amount of the protein comprising an IL-2 moiety is between about 0.004 mg/m² and 4 mg/m².

18. (canceled)

19. The method of claim 1, wherein the patient is a human.

20. A method of treating cancer comprising enhancing the immunostimulatory effect of IL-2 in a patient according to the method of claim 1.

21. (canceled)

22. The method of claim 1, further comprising the step of administering an anticancer vaccine.

23. (canceled)

24. The method of claim 1, wherein the first and second IL-2 moieties are mature human IL-2 moieties.

25-33. (canceled)

34. A method of stimulating effector cell function in a patient comprising administering to a patient an IL-2 fusion protein and an inhibitor of the interaction between IL-2 and an IL-2 receptor α subunit, wherein the inhibitor is an anti-CD25 antibody or an anti-IL-2 antibody, and wherein the inhibitor is administered in an amount effective to enhance the immunostimulatory effect of the IL-2 fusion protein.

35. (canceled)

36. The method of claim 34, wherein the immunostimulatory effect of the IL-2 fusion protein increases the population of NK cells, cytotoxic T-cells, or granulocytes.

37-51. (canceled)

52. The method of claim 1, wherein the protein comprising first and second IL-2 moieties is KS-IL-2.
